SEQUENCE LISTING (44 sequences)

SEQ ID NO: 1 - protein, 36 aa, Human immunodeficiency virus
YTSLIHSLIEESQNQQEKNEQELLELDKWASLWNWF

SEQ ID NO: 2 - protein, 39 aa, Artificial Sequence
  Synthetic peptide
WQEWEQKITALLEQAQIQQEKNEYELQKLDKWASLWEWF

SEQ ID NO: 3 - protein, 40 aa, Artificial Sequence
  Synthetic peptide
QLLSGIVQQQNNLLRAIEAQQHLLQLTVWGIKQLQARILA

SEQ ID NO: 4 - protein, 38 aa, Artificial Sequence
  Synthetic peptide
HTTWMEWDREINNYTSLIHSLIEESQNQQEKNEQELLE

SEQ ID NO: 5 - protein, 5 aa, Artificial Sequence
  Synthetic linker sequence
GGSGG

SEQ ID NO: 6 - protein, 5 aa, Artificial Sequence
  Synthetic linker sequence
GSSGG

SEQ ID NO: 7 - protein, 101 aa, Nostoc ellipsosporum
LGKFSQTCYNSAIQGSVLTSTCERTNGGYNTSSIDLNSVIENVDGSLKWQPSNFIETCRNTQLAGSSELAAECKTRAQQFVSTKINLDDHIANIDGTLKYE

SEQ ID NO: 8 - protein, 5 aa, Artificial Sequence
  Synthetic exemplary linker
GGGGS

SEQ ID NO: 9 - protein, 4 aa, Artificial Sequence
  Synthetic exemplary linker
GGGS

SEQ ID NO: 10 - protein, 17 aa, Artificial Sequence
  Stanniocalcin signal sequence
MLQNSAVLLLLVISASA

SEQ ID NO: 11 - protein, 22 aa, Artificial Sequence
  Consensus signal sequence
MPTWAWWLFLVLLLALWAPARG

SEQ ID NO: 12 - DNA, 56 bp, Artificial Sequence
  Synthetic oligonucleotide
ttaggcttaggtggttctggtggttccggtggttctggtggatccggtggttaata

SEQ ID NO: 13 - DNA, 57 bp, Artificial Sequence
  Synthetic oligonucleotide
agcttattaaccaccggatccaccagaaccaccggaaccaccagaaccacctaagcc

SEQ ID NO: 14 - DNA, 48 bp, Artificial Sequence
  Synthetic oligonucleotide
gatctttggataagagagacgctcacaagtccgaagtcgctcaccggt

SEQ ID NO: 15 - DNA, 50 bp, Artificial Sequence
  Synthetic oligonucleotide
ccttgaaccggtgagcgacttcggacttgtgagcgtctctcttatccaaa

SEQ ID NO: 16 - DNA, 48 bp, Artificial Sequence
  Synthetic oligonucleotide
gatctttggataagagagacgctcacaagtccgaagtcgctcatcgat

SEQ ID NO: 17 - DNA, 1782 bp, Homo sapiens
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaaatgtgccagtctccaaaaatttggagaaagagctttcaaagcatgggcagtggctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaggattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaactgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcacaaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataacatctacatttaaaagcatctcag

SEQ ID NO: 18 - protein, 585 aa, Homo sapiens [sequence omitted]

SEQ ID NO: 19 - DNA, 50 bp, Artificial Sequence
  Synthetic oligonucleotide
ccttgaatcgatgagcgacttcggacttgtgagcgtctctcttatccaaa

SEQ ID NO: 20 - DNA, 86 bp, Artificial Sequence
  Synthetic oligonucleotide
tcaaggacctaggtgaggaaaacttcaaggctttggtcttgatcgctttcgctcaatacttgcaacaatgtccattcgaagatcac

SEQ ID NO: 21 - DNA, 80 bp, Artificial Sequence
  Synthetic oligonucleotide
gtgatcttcgaatggacattgttgcaagtattgagcgaaagcgatcaagaccaaagccttgaagttttcctcacctaggt

SEQ ID NO: 22 - DNA, 85 bp, Artificial Sequence
  Synthetic oligonucleotide
gatctttggataagagaggtggatccggtggttccggtggttctggtggttccggtggtgacgctcacaagtccgaagtcgctca

SEQ ID NO: 23 - DNA, 85 bp, Artificial Sequence
  Synthetic oligonucleotide
ccggtgagcgacttcggacttgtgagcgtcaccaccggaaccaccagaaccaccggaaccaccggatccacctctcttatccaaa

SEQ ID NO: 24 - DNA, 47 bp, Artificial Sequence
  Synthetic oligonucleotide
gtgagatctttggataagagatggcaagaatgggaacaaaagattac

SEQ ID NO: 25 - DNA, 43 bp, Artificial Sequence
  Synthetic oligonucleotide
cacgagcttgttccaacaaagcagtaatcttttgttcccattc

SEQ ID NO: 26 - DNA, 59 bp, Artificial Sequence
  Synthetic oligonucleotide
gtgagctcaaattcaacaagaaaagaacgaatacgaattgcaaaagttggacaagtggg

SEQ ID NO: 27 - DNA, 63 bp, Artificial Sequence
  Synthetic oligonucleotide
cacggatccaccgaaccattcccacaaagaagcccacttgtccaacttttgcaattcgtattc

SEQ ID NO: 28 - DNA, 41 bp, Artificial Sequence
  Primer
gtgggatccggtggttggcaagaatgggaacaaaagattac

SEQ ID NO: 29 - DNA, 34 bp, Artificial Sequence
  Primer
cacaagcttattagaaccattcccacaaagaagc

SEQ ID NO: 30 - DNA, 20 bp, Artificial Sequence
  Primer
gtgccttggaatgctagttg

SEQ ID NO: 31 - DNA, 20 bp, Artificial Sequence
  Primer
cttaaacctaccaagcctcc

SEQ ID NO: 32 - DNA, 53 bp, Artificial Sequence
  Primer
ctctagatctttggataagagatacaccagcttaatacactccttaattgaag

SEQ ID NO: 33 - DNA, 44 bp, Artificial Sequence
  Primer
ccaccggatccaccaaaccaattccacaaacttgcccatttatc

SEQ ID NO: 34 - DNA, 53 bp, Artificial Sequence
  Primer
tggtggatccggtggttacaccagcttaatacactccttaattgaagaatcgc

SEQ ID NO: 35 - DNA, 51 bp, Artificial Sequence
  Primer
aattaagcttattaaaaccaattccacaaacttgcccatttatctaattcc

SEQ ID NO: 36 - DNA, 1986 bp, Artificial Sequence
  Synthetic DNA sequence of an N terminal T 1249 (GGS)4GG albumin fusion open reading frame
atgaagtgggttttcatcgtctccattttgttcttgttctcctctgcttactctagatctttggataagagatggcaagaatgggaacaaaagattactgctttgttggaacaagctcaaattcaacaagaaaagaacgaatacgaattgcaaaagttggacaagtgggcttctttgtgggaatggttcggtggatccggtggttccggtggttctggtggttccggtggtgacgctcacaagtccgaagtcgctcaccggttcaaggacctaggtgaggaaaacttcaaggctttggtcttgatcgctttcgctcaatacttgcaacaatgtccattcgaagatcacgtcaagttggtcaacgaagttaccgaattcgctaagacttgtgttgctgacgaatctgctgaaaactgtgacaagtccttgcacaccttgttcggtgataagttgtgtactgttgctaccttgagagaaacctacggtgaaatggctgactgttgtgctaagcaagaaccagaaagaaacgaatgtttcttgcaacacaaggacgacaacccaaacttgccaagattggttagaccagaagttgacgtcatgtgtactgctttccacgacaacgaagaaaccttcttgaagaagtacttgtacgaaattgctagaagacacccatacttctacgctccagaattgttgttcttcgctaagagatacaaggctgctttcaccgaatgttgtcaagctgctgataaggctgcttgtttgttgccaaagttggatgaattgagagacgaaggtaaggcttcttccgctaagcaaagattgaagtgtgcttccttgcaaaagttcggtgaaagagctttcaaggcttgggctgtcgctagattgtctcaaagattcccaaaggctgaattcgctgaagtttctaagttggttactgacttgactaaggttcacactgaatgttgtcacggtgacttgttggaatgtgctgatgacagagctgacttggctaagtacatctgtgaaaaccaagactctatctcttccaagttgaaggaatgttgtgaaaagccattgttggaaaagtctcactgtattgctgaagttgaaaacgatgaaatgccagctgacttgccatctttggctgctgacttcgttgaatctaaggacgtttgtaagaactacgctgaagctaaggacgtcttcttgggtatgttcttgtacgaatacgctagaagacacccagactactccgttgtcttgttgttgagattggctaagacctacgaaactaccttggaaaagtgttgtgctgctgctgacccacacgaatgttacgctaaggttttcgatgaattcaagccattggtcgaagaaccacaaaacttgatcaagcaaaactgtgaattgttcgaacaattgggtgaatacaagttccaaaacgctttgttggttagatacactaagaaggtcccacaagtctccaccccaactttggttgaagtctctagaaacttgggtaaggtcggttctaagtgttgtaagcacccagaagctaagagaatgccatgtgctgaagattacttgtccgtcgttttgaaccaattgtgtgttttgcacgaaaagaccccagtctctgatagagtcaccaagtgttgtactgaatctttggttaacagaagaccatgtttctctgctttggaagtcgacgaaacttacgttccaaaggaattcaacgctgaaactttcaccttccacgctgatatctgtaccttgtccgaaaaggaaagacaaattaagaagcaaactgctttggttgaattggtcaagcacaagccaaaggctactaaggaacaattgaaggctgtcatggatgatttcgctgctttcgttgaaaagtgttgtaaggctgatgataaggaaacttgtttcgctgaagaaggtaagaagttggtcgctgcttcccaagctgctttgggtttg

SEQ ID NO: 37 - protein, 662 aa, Artificial Sequence
  Synthetic amino acid sequence of an N terminal T 1249 (GGS)4GG  albumin fusion protein
MKWVFIVSILFLFSSAYSRSLDKRWQEWEQKITALLEQAQIQQEKNEYELQKLDKWASLWEWFGGSGGSGGSGGSGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 38 - DNA, 1986 bp, Artificial Sequence
  Synthetic DNA sequence of a C terminal albumin (GGS)4GG T 1249 fusion open reading frame
atgaagtgggtaagctttatttcccttctttttctctttagctcggcttattccaggagcttggataaaagagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggtggttctggtggttccggtggttctggtggatccggtggttggcaagaatgggaacaaaagattactgctttgttggaacaagctcaaattcaacaagaaaagaacgaatacgaattgcaaaagttggacaagtgggcttctttgtgggaatggttc

SEQ ID NO: 39 - protein, 662 aa, Artificial Sequence
  Synthetic amino acid sequence of a C terminal albumin (GGS)4GG T 1249 fusion protein
MKWVSFISLLFLFSSAYSRSLDKRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSGGSGGSGGSGGWQEWEQKITALLEQAQIQQEKNEYELQKLDKWASLWEWF

SEQ ID NO: 40 - DNA, 1977 bp, Artificial Sequence
  Synthetic DNA sequence of an N terminal T 20 (GGS)4GG albumin fusion open reading frame
atgaagtgggttttcatcgtctccattttgttcttgttctcctctgcttactctagatctttggataagagatacaccagcttaatacactccttaattgaagaatcgcaaaaccagcaagaaaagaatgaacaagaattattggaattagataaatgggcaagtttgtggaattggtttggtggatccggtggttccggtggttctggtggttccggtggtgacgctcacaagtccgaagtcgctcaccggttcaaggacctaggtgaggaaaacttcaaggctttggtcttgatcgctttcgctcaatacttgcaacaatgtccattcgaagatcacgtcaagttggtcaacgaagttaccgaattcgctaagacttgtgttgctgacgaatctgctgaaaactgtgacaagtccttgcacaccttgttcggtgataagttgtgtactgttgctaccttgagagaaacctacggtgaaatggctgactgttgtgctaagcaagaaccagaaagaaacgaatgtttcttgcaacacaaggacgacaacccaaacttgccaagattggttagaccagaagttgacgtcatgtgtactgctttccacgacaacgaagaaaccttcttgaagaagtacttgtacgaaattgctagaagacacccatacttctacgctccagaattgttgttcttcgctaagagatacaaggctgctttcaccgaatgttgtcaagctgctgataaggctgcttgtttgttgccaaagttggatgaattgagagacgaaggtaaggcttcttccgctaagcaaagattgaagtgtgcttccttgcaaaagttcggtgaaagagctttcaaggcttgggctgtcgctagattgtctcaaagattcccaaaggctgaattcgctgaagtttctaagttggttactgacttgactaaggttcacactgaatgttgtcacggtgacttgttggaatgtgctgatgacagagctgacttggctaagtacatctgtgaaaaccaagactctatctcttccaagttgaaggaatgttgtgaaaagccattgttggaaaagtctcactgtattgctgaagttgaaaacgatgaaatgccagctgacttgccatctttggctgctgacttcgttgaatctaaggacgtttgtaagaactacgctgaagctaaggacgtcttcttgggtatgttcttgtacgaatacgctagaagacacccagactactccgttgtcttgttgttgagattggctaagacctacgaaactaccttggaaaagtgttgtgctgctgctgacccacacgaatgttacgctaaggttttcgatgaattcaagccattggtcgaagaaccacaaaacttgatcaagcaaaactgtgaattgttcgaacaattgggtgaatacaagttccaaaacgctttgttggttagatacactaagaaggtcccacaagtctccaccccaactttggttgaagtctctagaaacttgggtaaggtcggttctaagtgttgtaagcacccagaagctaagagaatgccatgtgctgaagattacttgtccgtcgttttgaaccaattgtgtgttttgcacgaaaagaccccagtctctgatagagtcaccaagtgttgtactgaatctttggttaacagaagaccatgtttctctgctttggaagtcgacgaaacttacgttccaaaggaattcaacgctgaaactttcaccttccacgctgatatctgtaccttgtccgaaaaggaaagacaaattaagaagcaaactgctttggttgaattggtcaagcacaagccaaaggctactaaggaacaattgaaggctgtcatggatgatttcgctgctttcgttgaaaagtgttgtaaggctgatgataaggaaacttgtttcgctgaagaaggtaagaagttggtcgctgcttcccaagctgctttgggtttg

SEQ ID NO: 41 - protein, 659 aa, Artificial Sequence
  Synthetic amino acid sequence of an N terminal T 20 (GGS)4GG albumin fusion protein
MKWVFIVSILFLFSSAYSRSLDKRYTSLIHSLIEESQNQQEKNEQELLELDKWASLWNWFGGSGGSGGSGGSGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 42 - DNA, 1977 bp, Artificial Sequence
  Synthetic DNA sequence of a C terminal albumin (GGS)4GG T 20 fusion open reading frame
atgaagtgggtaagctttatttcccttctttttctctttagctcggcttattccaggagcttggataaaagagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggtggttctggtggttccggtggttctggtggatccggtggttacaccagcttaatacactccttaattgaagaatcgcaaaaccagcaagaaaagaatgaacaagaattattggaattagataaatgggcaagtttgtggaattggttt

SEQ ID NO: 43 - protein, 659 aa, Artificial Sequence
  Synthetic amino acid sequence of a C terminal albumin (GGS)4GG T20 fusion protein
MKWVSFISLLFLFSSAYSRSLDKRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSGGSGGSGGSGGYTSLIHSLIEESQNQQEKNEQELLELDKWASLWNWF

SEQ ID NO: 44 - protein, 14 aa, Artificial Sequence
  Synthetic polypeptide linker
GGSGGSGGSGGSGG

(19) United States
(12) Patent Application Publication
(10) Pub. No.: US 2009/0227775 A1
(43) Pub. Date: Sep. 10, 2009

(54) HIV Inhibiting Proteins

(75) Inventors: Hans-Peter HAUSER, Marburg (DE); Thomas Weimer, Gladenbach (DE); Darrell Sleep, Nottingham (GB)

Correspondence Address: Ballard Spahr Andrews & Ingersoll, LLP, SUITE 1000, 999 PEACHTREE STREET, ATLANTA, GA, 30309-3915, US

(73) Assignee: NOVOZYMES BIOPHARMA UK LIMITED, Nottingham (GB)

(21) Appl. No.: 12/401,241

(22) Filed: Mar. 10, 2009

Related U.S. Application Data
(63) Continuation of application No. 10/503,832, filed on Sep. 26, 2005, now abandoned.
(60) Provisional application No. 60/355,547, filed on Feb. 7, 2002.

(51) Int. Cl. C07K 14/76

(52) U.S. Cl. 530/362

(57) ABSTRACT

The invention relates to proteins comprising HIV fusion inhibiting peptides, such as T-20 and/or T-1249 peptides (including, but not limited to, fragments and variants thereof), which exhibit anti-retroviral activity, fused to albumin (including, but not limited to fragments or variants of albumin). These fusion proteins exhibit extended shelf-life and/or extended or therapeutic activity.

### CROSS-REFERENCE TO RELATED APPLICATIONS

This application is a continuation application of U.S. application Ser. No. 10/503,832 which is a National Stage application based on International Application No. PCT/IB03/00434, filed Feb. 7, 2003, which claims priority to U.S. Provisional Application No. 60/355,547, filed Feb. 7, 2002, the disclosures of which are incorporated herein by reference.

### FIELD OF THE INVENTION

The invention relates to fields of HIV fusion inhibitors and albumin fusion proteins.

### BACKGROUND OF THE INVENTION

### Background

At the end of 2001, there were an estimated 940,000 adults and children living with HIV/AIDS in the United States and Canada. The adult prevalence rate for this region was 0.6 percent, with women accounting for 20 percent of HIV-positive adults. During 2001, 45,000 adults and children in the region became newly infected with HIV (UNAIDS AIDS Epidemic Update December 2001).

Significant progress has been made over the last several years in the development of antiretroviral therapy to fight Human Immunodeficiency Virus (HIV), primarily targeting viral replication by interfering with the reverse transcription process and maturation of the virus. New classes of drugs are however required to overcome problems of drug tolerability and toxic effects, latent viral reservoirs, and drug resistance. There is a need for safer treatments with improved dosing regimens to promote better compliance with anti-retroviral therapy. These treatments must have an acceptable risk/benefit profile and should be able to be used concomitantly with other anti-retroviral therapies. A promising approach for drug development is interference with HIV entry.

The Envelope glycoprotein of HIV-1 is produced as a gp160 precursor that is embedded in the membrane bilayer surrounding the virion core. Nearly 50% of the glycoprotein's 160 kDa weight is composed of N-linked carbohydrates. Env is proteolytically cleaved during synthesis into an exterior gp120 subunit and a transmembrane gp41 subunit that are non-covalently associated. The gp120 subunit is responsible for attachment to cellular receptors and, upon binding to its receptors, undergoes a conformational change. The conformational change in gp120 leads to the exposure of the fusion peptide of gp41, a hydrophobic patch of amino acids that directly mediates membrane fusion. Upon triggering, gp41 undergoes a massive conformational change in which two α-helices form coiled coils that help insert the fusion peptide and form the fusion pore. Interference with these conformational changes by using peptide mimics of these helices or with small molecules that bind to cnicial helical structures is proving to be an effective method of inhibiting the virus from entering its target cell. (For reviews, see Chan D C and Kim P S (1998) Cell 93:681-684; Doranz B J (2000) Emerging Therapeutic Targets 4:423-437; LaBranche C C (2001) Antiviral Research 50:95-115). Env reaches the surface of the cell as a trimer of gp120/gp41 subunits and facilitates fusion by forming a pore between the viral membrane and the cell membrane.

T-20, also known as DP-178, is a conserved 36 amino acid peptide from the C-peptide of gp41. It binds to the prehairpin intermediate and inhibits further conformational changes in gp41, thereby blocking viral entry into the target cell (Wild C T et al. (1994) Proc. Natl. Acad. Sci. 91:9770-9774). It has been shown to be active against several clades of HIV-1, however, it does not inhibit HIV-2 or SIV. It displays antiviral activity in cell-cell fusion assays in the low nanomolar range (0.2-2 nM). T-20 corresponds to amino acids (aa) 638 to 673 (36 aa) of the HIV-1 gp41 protein.

T-1249 is another fusion inhibitor similar in design to T-20 but active against HIV-1, HIV-2, and SIV and including an enhancer sequence (U.S. Pat. No. 6,258,782). The enhancer sequence is derived from gp41 and is claimed to improve pharmacodynamic properties of any peptide, if attached. T-1249 is 39 amino acids in length, partially homologous to T-20, but with amino acid exchanges and additional gp41 amino acid sequences and with the potential to treat T-20 resistant viruses. In addition, T-1249 has a longer half-life than T-20 in primates (twofold increased AUC), which may allow for once-daily dosing.

5-Helix, which is derived from amino acid positions 558 to 678 of gp41 of HIV-1, contains 5 of the 6 helices (three N- and two C-helices) that make up the core of the gp41 trimer-of-hairpins structure, connected by short peptide linkers. The vacancy of the third C-peptide is expected to create a binding site for the carboxyterminal region of gp41. If this region is accessible (at least transiently) before formation of the trimer-of hairpins, the binding of 5-Helix is expected to prevent the conformational changes associated with the fusion event and thereby prevent infection of the cell (Root M I (2001) Science 291:884-888).

5-Helix, which was made by bacterial expression and was stable under physiological conditions in vitro, has been shown to potently inhibit HIV-1 membrane fusion in the nanomolar range, as measured by viral infectivity and cell-cell fusion assays. From this, it has been concluded that binding of the C-peptide is the key determinant in antiviral activity of 5-Helix. 5-Helix has been shown to inhibit HIV-1 infection of isolates from clacks A, B and D, demonstrating the conserved interface between N- and C-terminal regions within the gp411 trimer-of-hairpins.

Cyanovirin-N is a small protein isolated from Nostoc ellipsosporum, which binds to glycostructures on gp120. (Boyd et al. (1997) An-timid-ob. Agents Chemother. 41:1521-1530; Gustafson et al. (1997) Biochem. Biophys. Res. Comm. 238:223-228).

Clinical trials have been performed for T-20 and T-1249. For T-20, a typical dose of 50 mg twice daily was employed. The plasma half-life was determined to be about 1.8 hours. T-1249 has a somewhat longer half-life than T-20 in primates (twofold increased AUC).

The expected continuation of IIAART use in both the long and short term and the growing susceptibility to therapy failure through drug resistance suggest that there is a real role for multiple innovative anti-retroviral therapies including entry inhibitors. HIV fusion inhibitors, such as T-20 and T-1249, provide a new treatment principle in addition to the classical protease and reverse transcriptase inhibitors. The albumin fusion technology, if able to extend plasma half-life and bioavailability significantly, could provide a once-weekly dosing and could significantly increase the acceptability of a parenteral HIV drug for first-line treatment. Products like T-20 and T-1249 albumin fusions (reg., SEQ ID Nos. 40-43 and 36-39) due to their improved side effect profile may improve regimen tolerability for some patients. As peptides like T-20 and T-1249 are of hydrophobic nature their fusion to albumin improves their solubility which should also result in an increase of bioavailability and should allow for higher concentrated formulations.

### SUMMARY OF THE INVENTION

The invention relates to proteins comprising HIV fusion inhibiting peptides (including, but not limited to, peptides binding to the HIV env protein or peptides derived from the HIV env protein), fused to albumin or fragments or variants thereof. These fusion proteins are herein collectively referred to as “albumin fusion proteins of the invention.” These fusion proteins of the invention exhibit extended in vivo half-life and/or extended or therapeutic activity.

The invention encompasses therapeutic albumin fusion proteins, compositions, pharmaceutical compositions, formulations and kits. The invention also encompasses nucleic acid molecules encoding the albumin fusion proteins of the invention, as well as vectors containing these nucleic acids, host cells transformed with these nucleic acids and vectors, and methods of making the albumin fusion proteins of the invention using these nucleic acids, vectors, and/or host cells.

The invention also relates to compositions and methods for inhibiting HIV-induced cell fusion. The invention further relates to compositions and methods for inhibiting HIV transmission to uninfected cells and for preventing and/or treating HIV related diseases.

### BRIEF DESCRIPTION OF THE DRAWINGS

FIG. 1. DNA sequence of an N-terminal T-1244-(GGS)₄GG-albumin fusion open reading frame (SEQ ID No.: 36). (This DNA sequence encodes the primary translation product and, therefore, the first 72 nucleotides encode a 24 amino acid leader sequence which is removed during secretion from yeast in the examples herein).

FIG. 2. Amino acid sequence of an N-terminal T-1249-(GGS)₄GG-albumin fusion protein (SEQ ID No.: 39). (This amino acid sequence represents the primary translation product of the DNA sequence shown in FIG. 1 and, therefore, includes a 24 amino acid leader sequence which is removed during secretion in yeast. Thus, the protein sequence does not represent the sequence of the protein used in the viral inhibition examples herein).

FIG. 3. DNA sequence of a C-terminal albumin-(GGS)₄GG-T-1249 fusion open reading frame (SEQ ID No.: 38). (This DNA sequence encodes the primary translation product and, therefore, the first 72 nucleotides encode a 24 amino acid leader sequence which is removed during secretion from yeast in the examples herein).

FIG. 4. Amino acid sequence of a C-terminal albumin-(GGS)₄GG-T-1249 fusion protein (SEQ ID No. 39). (This amino acid sequence represents the primary translation product of the DNA sequence shown in FIG. 3 and, therefore, includes a 24 amino acid leader sequence which is removed during secretion in yeast. Thus, the protein sequence does not represent the sequence of the protein used in the viral inhibition examples herein).

FIG. 5. DNA sequence of an N-terminal T-20-(GGS)₄GG-albumin fusion open reading frame (SEQ ID No. 40). (This DNA sequence encodes the primary translation product and, therefore, the first 72 nucleotides encode a 24 amino acid leader sequence which is removed during secretion from yeast in the examples herein)

FIG. 6. Amino acid sequence of an N-terminal T-20-(GGS)₄GG-albumin fusion protein (SEQ ID No. 41). (This amino acid sequence represents the primary translation product of the DNA sequence shown in FIG. 5 and, therefore, includes a 24 amino acid leader sequence which is removed during secretion in yeast. Thus, the protein sequence does not represent the sequence of the protein used in the viral inhibition examples herein).

FIG. 7. DNA sequence of a C-terminal albumin-(GGS)₄GG-T-20 fusion open reading frame (SEQ ID No. 42). (This DNA sequence encodes the primary translation product and, therefore, the first 72 nucleotides encode a 24 amino acid leader sequence which is removed during secretion from yeast in the examples herein).

FIG. 8. Amino acid sequence of a C-terminal albumin-(GGS)₄GG-T-20 fusion protein (SEQ ID No. 43). (This amino acid sequence represents the primary translation product of the DNA sequence shown in FIG. 7 and, therefore, includes a 24 amino acid leader sequence which is removed during secretion in yeast. Thus, the protein sequence does not represent the sequence of the protein used in the viral inhibition examples herein).

FIG. 9. 4-12% gradient SDS non-reducing gel with T-20 albumin fusions: (A) Colloidal Blue gel; (B) Anti-HSA Western blot.

FIG. 10. Antiviral activity of T-20 albumin fusions in a cell-cell fusion assay in dependence of expression in a pmtl gene deficient yeast strain. PATT1, wild-type; pmtl, deficient strain.

FIG. 11. 4-1.2% gradient SDS non-reducing gel with C-Terminal T-1249 albumin fusion: (A) Colloidal Blue gel; (B) Anti-HSA Western blot.

FIG. 12. Antiviral activity of a C-terminal T-1249 albumin fusion protein.

FIG. 13. Pharmacokinetic study results for C-terminal T-20 albumin fusion protein.

FIG. 14 (A-D). Amino acid sequence of a mature form of human albumin (SEQ ID NO:18) and a polynucleotide encoding it (SEQ ID NO:17).

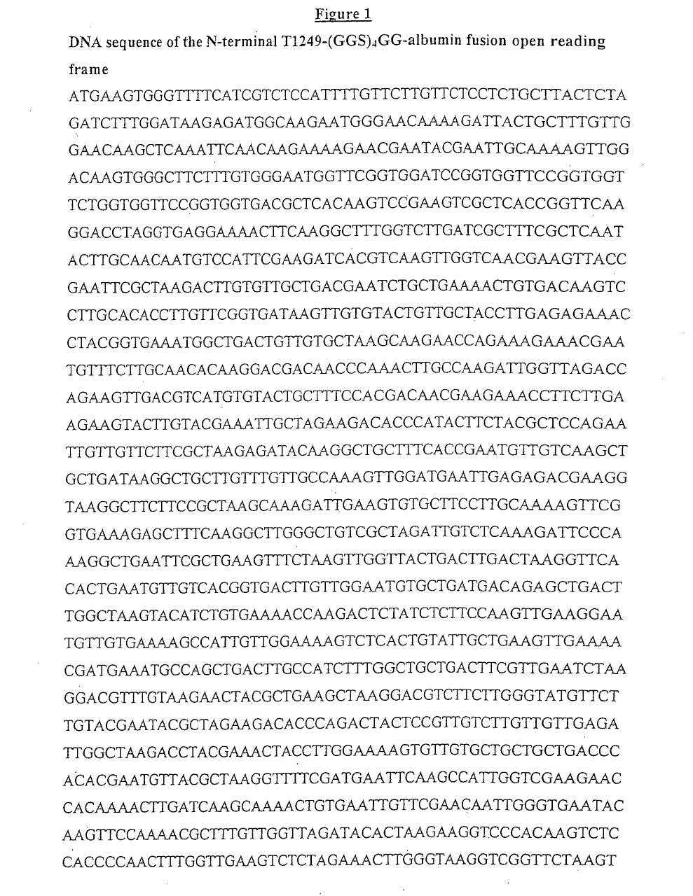

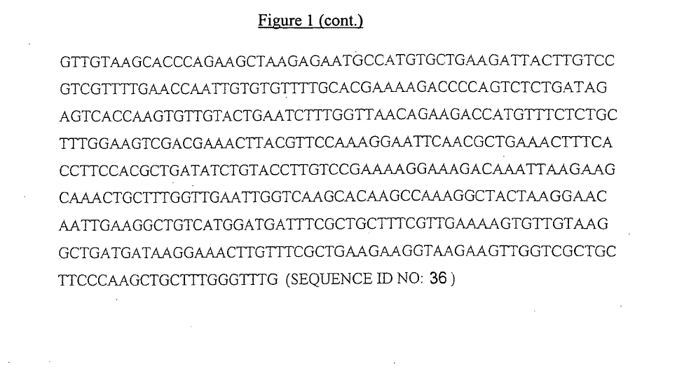

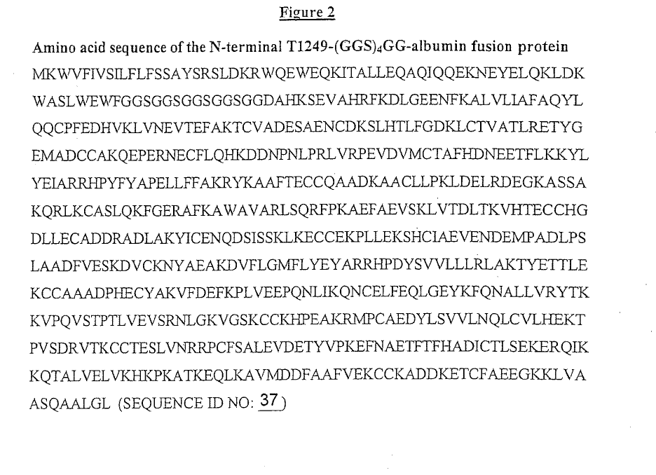

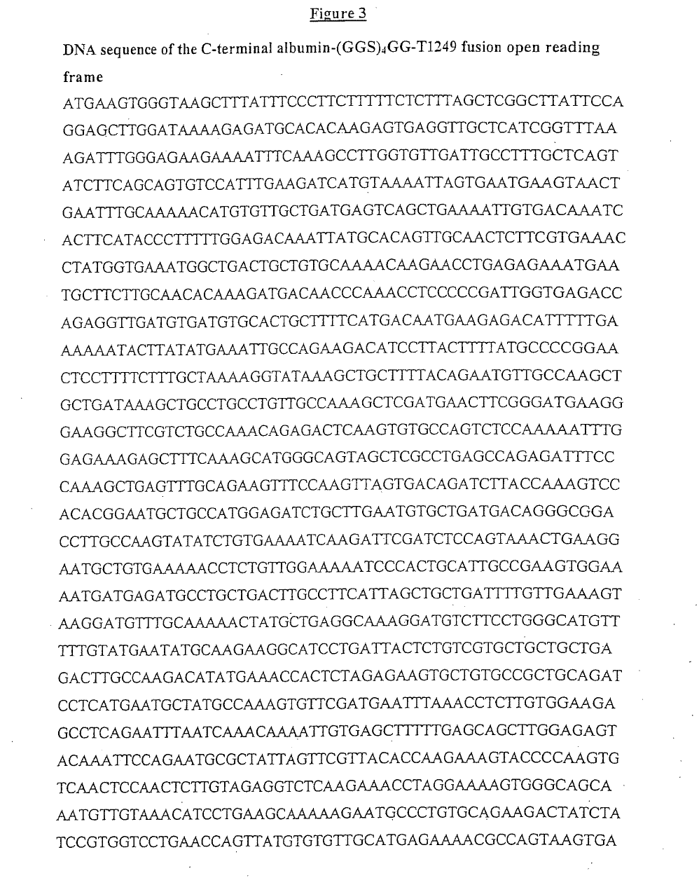

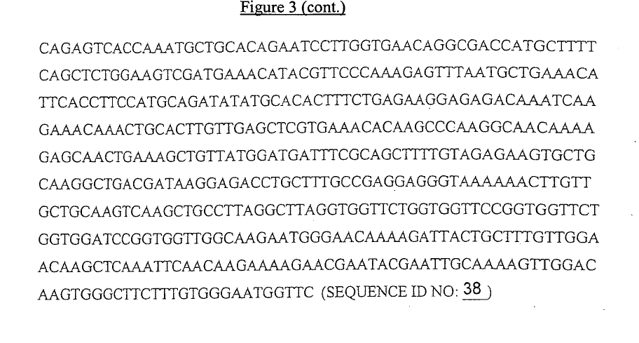

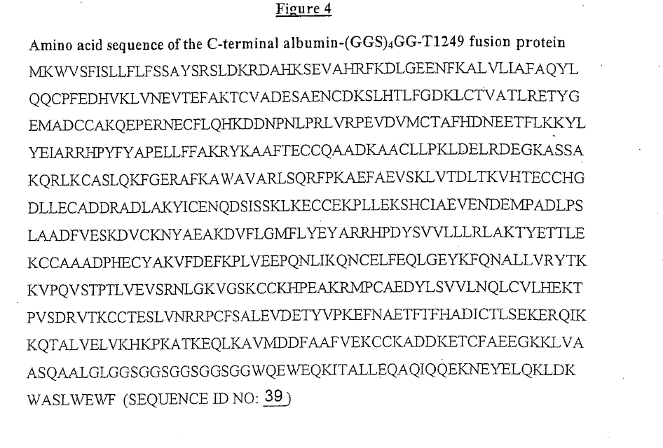

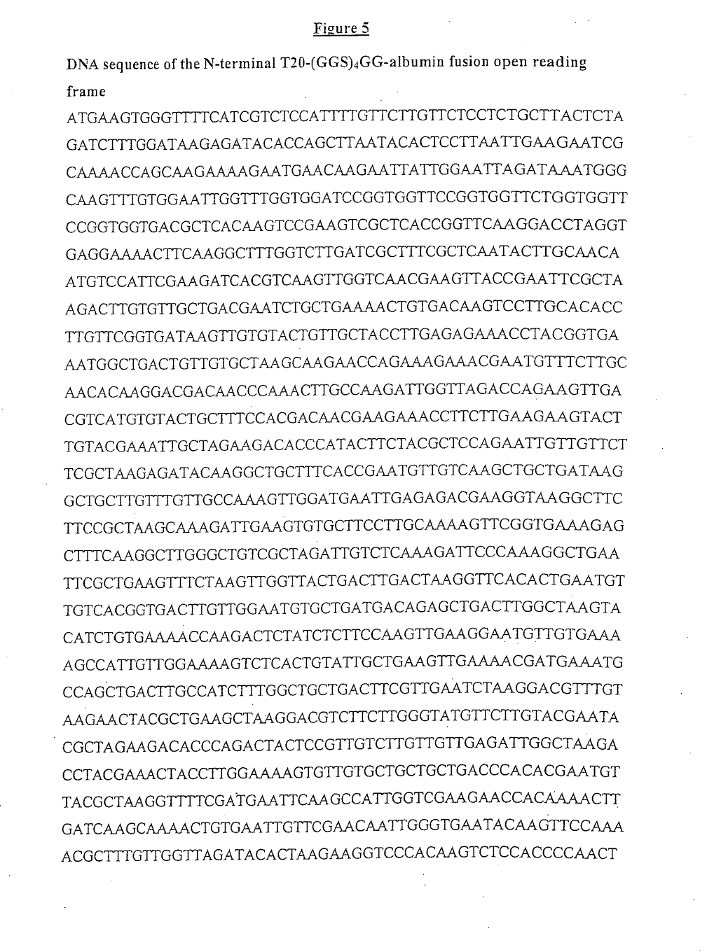

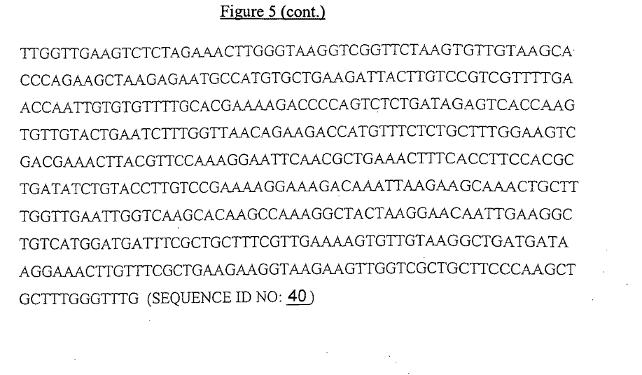

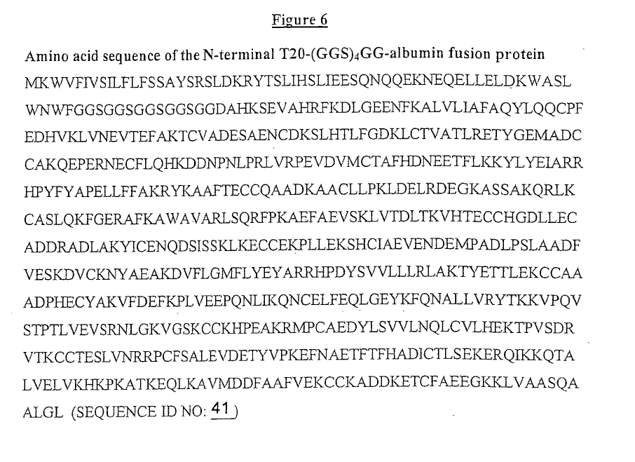

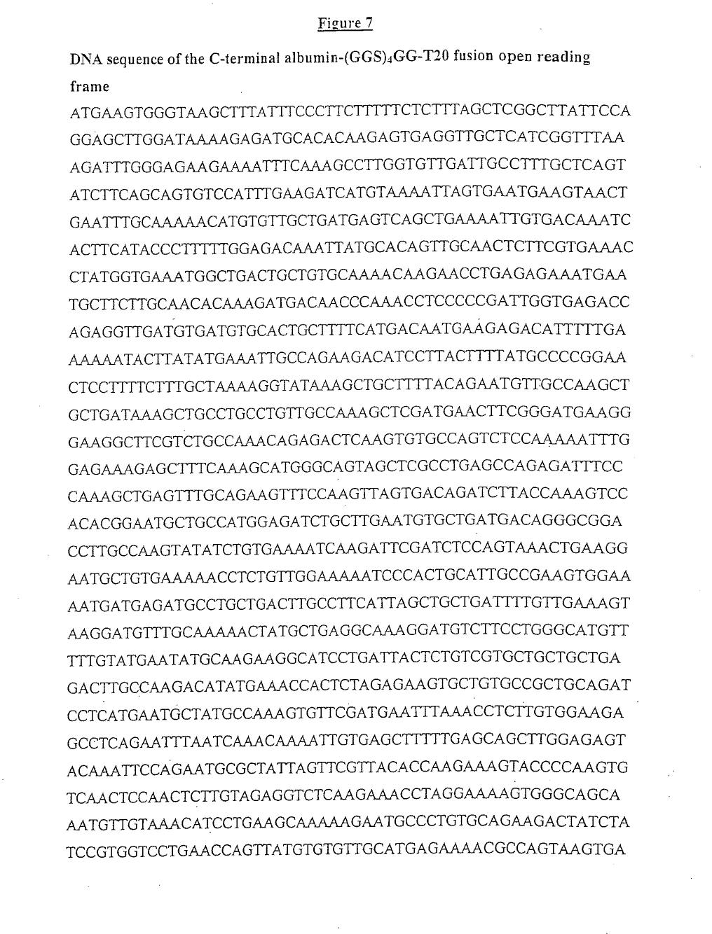

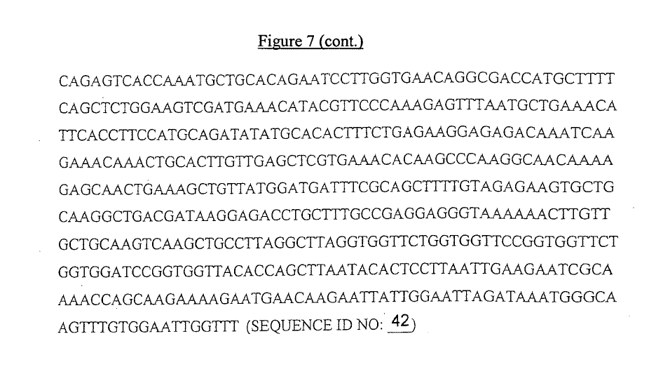

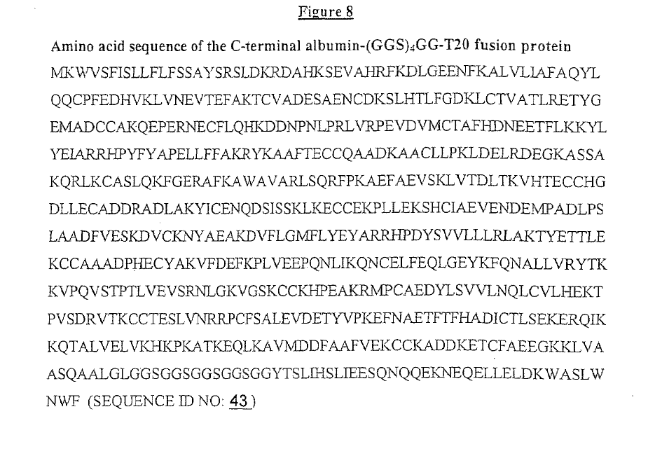

### DETAILED DESCRIPTION OF THE INVENTION

The present invention relates to fusion proteins comprising albumin coupled to HIV fusion inhibiting peptides. Such peptides include, but are not limited to, peptides binding to the HIV env protein or peptides derived from the HIV env protein, including peptides binding to HIV gp41 or peptides derived from HIV gp41. These peptides include T-20, T-1249, 5-Helix or cyanovirin-N, or fragments or variants thereof, which have HIV-fusion inhibiting properties.

The terms “protein” and “peptide” as used herein are non-limiting and include proteins and polypeptides as well as peptides.

The present invention also relates to bifunctional (or multifunctional) fusion proteins in which albumin is coupled to two (or more) HIV fusion inhibiting peptides, optionally different HIV fusion inhibiting peptides. Such bifunctional (or multifunctional) fusion proteins having different HIV fusion inhibiting peptides are expected to have an improved drug resistance profile as compared to an albumin fusion protein comprising only one type of HIV fusion inhibiting peptide in that the generation of drug-resistant mutant HIV strains would significantly be delayed. Such bifunctional (or multifunctional) fusion proteins may also exhibit synergistic anti-HIV effects, as compared to an albumin fusion protein comprising only one type of HIV fusion inhibiting peptide (although it is noted that 5-Helix and C-peptides have been shown to be antagonistic (Root et al. 2001)).

The present invention also relates to fusion proteins in which one (or more) HIV fusion inhibiting peptides, optionally different HIV fusion inhibiting peptides, or fragments or variants thereof, is coupled to two albumin molecules, or fragments or variants thereof, which could be the same or different.

Furthermore, chemical entities may be covalently attached to the fusion proteins of the invention or used in combinations to enhance a biological activity or to modulate a biological activity.

The albumin fusion proteins of the present invention are expected to prolong the half-life of the HIV fusion inhibiting peptide in vivo. The in vitro or in vivo half-life of said albumin-fused peptide is extended 2-fold, or 5-fold, or more, over the half-life of the peptide lacking the linked albumin. Furthermore, due at least in part to the increased half-life of the peptide, the albumin fusion proteins of the present invention are expected to reduce the frequency of the dosing schedule of the therapeutic peptide. The dosing schedule frequency is reduced by at least one-quarter, or by at least one-half, or more, as compared to the frequency of the dosing schedule of the therapeutic peptide lacking the linked albumin.

The albumin fusion proteins of the present invention prolong the shelf-life of the peptide, and/or stabilize the peptide and/or its activity in solution (or in a pharmaceutical composition) in vitro and/or in vivo. These albumin-fusion proteins, which may be therapeutic agents, are expected to reduce the need to formulate protein solutions with large excesses of carrier proteins (such as albumin, unfused) to prevent loss of proteins due to factors such as nonspecific binding.

The present invention also encompasses nucleic acid molecules encoding the albumin fusion proteins as well as vectors containing these nucleic acids, host cells transformed with these nucleic acids vectors, and methods of malting the albumin fusion proteins of the invention using these nucleic acids, vectors, and/or host cells. The present invention further includes transgenic organisms modified to contain the nucleic acid molecules of the invention, optionally modified to express the albumin fusion proteins encoded by the nucleic acid molecules.

The present invention also encompasses pharmaceutical formulations comprising an albumin fusion protein of the invention and a pharmaceutically acceptable diluent or carrier. Such formulations may be in a kit or container. Such kit or container may be packaged with instructions pertaining to the extended shelf-life of the protein. Such formulations may be used in methods of preventing, treating, ameliorating or diagnosing HIV infection or a HIV-related disease, disease symptom or a related disorder in a patient, such as a mammal, or a human, comprising the step of administering the pharmaceutical formulation to the patient.

The invention also encompasses a method for potentially minimizing side effects (e.g., injection site reaction, headache, nausea, fever, increased energy levels, rash asthenia, diarrhea, dizziness, allergic reactions, abnormally low neutrophil levels) associated with the treatment of a mammal with HIV fusion inhibiting peptide in moderately higher concentrations comprising administering an albumin-fused HIV fusion inhibiting peptide of the invention to said mammal.

The present invention encompasses a method of preventing, treating or ameliorating HIV infection and/or a disease or disorder caused by HIV infection comprising administering to a mammal, in which such prevention treatment, or amelioration is desired an albumin fusion protein of the invention that comprises a HIV fusion inhibiting peptide (or fragment or variant thereof) in an amount effective to treat, prevent or ameliorate the disease or disorder. In the present invention, the HIV fusion inhibiting peptide, such as T-20 and/or T-1249, is also called the “Therapeutic protein”.

The present invention encompasses albumin fusion proteins comprising a T-20 and/or T-1249 peptide or multiple copies of monomers of T-20 and/or T-1249 (including fragments and variants thereof) fused to albumin or multiple copies of albumin (including fragments and variants thereof).

The present invention also encompasses a method for extending the half-life of HIV T-20 and/or T-1249 peptide in a mammal. The method entails linking HIV T-20 and/or T-1249 peptide to an albumin to form albumin-fused HIV T-20 and/or T-1249 peptide and administering the albumin-fused HIV T-20 and/or T-1249 peptide to a mammal. Typically, the half-life of the albumin-fused HIV T-20 and/or T-1249 peptide may be extended by at least 2-fold, 5-fold, 10-fold, 20-fold, 30-fold, 40-fold or at least 50-fold over the half-life of the HIV T-20 and/or T-1249 peptide lacking the linked albumin.

Exemplified herein are fusion proteins comprising albumin fused to T-20 and/or T-1249 which exhibit anti-viral activity. Such anti-viral activity includes, but is not limited to, the inhibition of HIV transmission to uninfected CD-4⁺ cells. Further, the invention relates to the use of such fusion proteins comprising albumin fused to T-20 and/or T-1249 as inhibitors of human and non-human retroviral, especially HIV, transmission to uninfected cells.

The present invention also includes an improved method of manufacturing a Therapeutic moiety as compared to what is available in the art. For example, the present invention provides an enhanced means of manufacturing a protein with the active moiety T-20 or T-1249 as compared to the complex chemical synthesis method currently available in the art. (See, e.g., SCRIP Magazine, September 2002, pp. 7-10 and WO 99/48513 “Methods and Compositions for Peptide Synthesis”)

Various aspects of the present invention are discussed in further detail below.

T-20

T-20 (also known as DP-178) is a peptide with the amino acid sequence YTSLIHSLIEESQNQQEKNEQELLELDKWASLWNWF (SEQ ID NO:1) corresponding to amino acid residues 638 to 673 of the transmembrane protein gp41 from the HIV-1_{LAI} isolate. A T-20 peptide useful in the present invention includes fragments or variants thereof, such as any molecule which is an analog, homolog, fragment, or a derivative of naturally occurring HIV T-20 peptide, such as those described in U.S. Pat. No. 6,133,418 and WO 94/28920 and the other patents and references listed in Table 1 herein which are specifically incorporated by reference herein. The HIV T-20 peptide-useful in the present invention need only possess a single biological activity of the HIV T-20 peptide of SEQ ID NO:1.

The T-20 peptides useful in the invention exhibit antiviral activity, and may, further, possess additional advantageous features, such as, for example, increased bioavailability, and/or stability, or reduced host immune recognition.

When T-20 (or a fragment or variant thereof) is to be expressed in yeast which is capable of O-glycosylation, any serines or threonines may be modified or otherwise decreased in number to minimize the effect of O-glycosylation or the biological activity of T-20 (or a fragment or variant thereof). Alternatively, or in addition, use of a yeast strain which underglycosylates (i.e., which is deficient in O-glycosylation) may be used.

T-1249

The amino acid sequence of T-1249 is WQEWEQKITALLEQAQIQQEKNEYELQKLDKWASLWEWF (SEQ ID NO:2). See, e.g., U.S. Pat. No. 6,258,782 and WO 99/59615. Active fragments and variants thereof which are useful in the albumin fusion proteins of the present invention can be identified using methods known in the art, including those described in the patents and references listed in Table 1, which are incorporated by reference herein.

5-Helix

5-Helix is a designed protein in which three N-peptide segments (N40) and two C-peptide segments (C38) are alternately linked (N-C-N-C-N) using short Gly/Ser peptide sequences. The sequences of each segment in single-letter amino acid code are: N40, QLLSGIVQQQNNLLRAIEAQQHLLQLTVWGIKQLQARILA (SEQ ID NO:3); C38, HTTWMEWDREINNYTSLIHSLIEESQ-NQQEKNEQELLE (SEQ ID NO:4); N-to-C linker, GGSGG (SEQ ID NO:5); and C-to-N linker, GSSGG (SEQ DD NO:6) (Root et al). Active fragments and variants thereof which are useful in the albumin fusion proteins of the present invention can be identified in the manner described in the patents and references listed in Table 1, which are incorporated by reference herein.

Cyanovirin-N

The amino acid sequence of Cyanovirin-N is LGKFSQTCYNSAIQGSVLTSTCERTNGGYNTSSIDLNSVIENVDGSLKWQPSNFIE TCRNTQLAGSSELAAECKTRAQQFVSTKINLDDMIANIDGTLKYE (SEQ ID NO:7) (Gustafson et al.). Active fragments and variants thereof which are useful in the albumin fusion proteins of the present invention can be identified in the manner described in the patents and references listed in Table 1, which are incorporated by reference herein.

Albumin

The terms, human serum albumin (HSA) and human albumin (HA) are used interchangeably herein. The terms, “albumin and “serum albumin” are broader, and encompass human serum albumin (and fragments and variants thereof) as well as albumin from other species (and fragments and variants thereof).

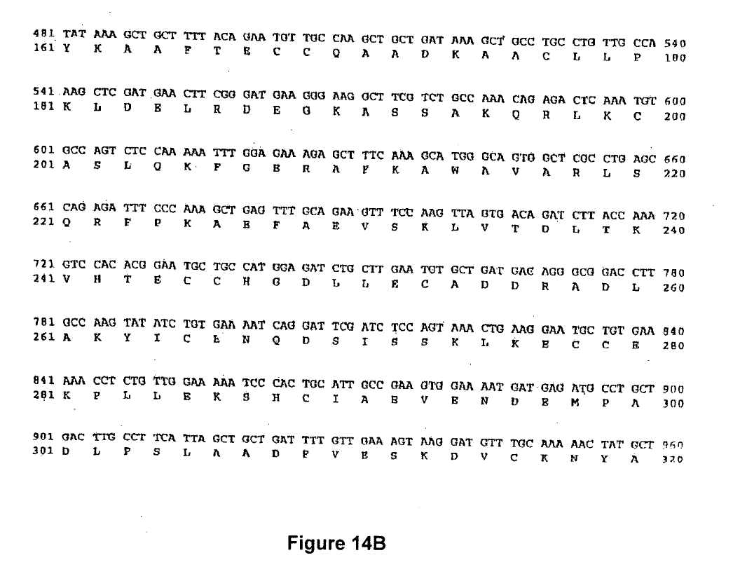

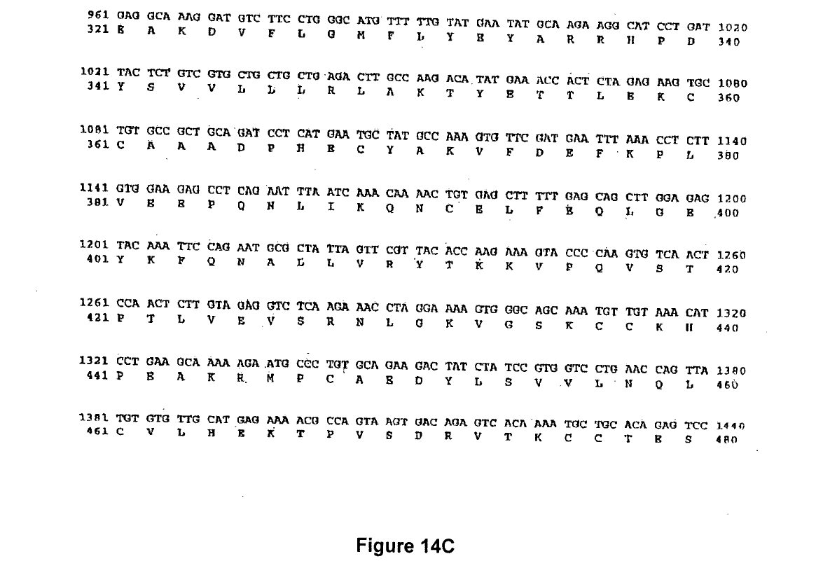

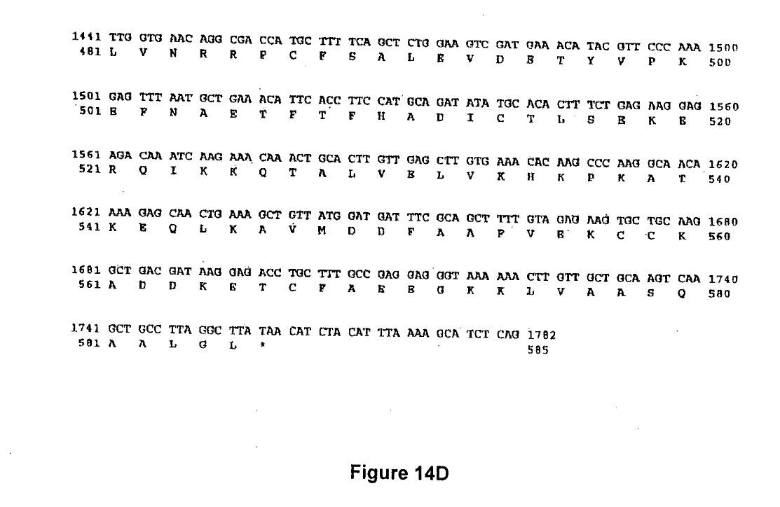

As used herein, “albumin” refers collectively to albumin protein or amino acid sequence, or an albumin fragment or valiant, having one or more functional activities (e.g., biological activities) of albumin. In particular, “albumin” refers to human albumin or fragments thereof (see EP 201 239, EP 322 094 WO 97/24445, WO95/23857) especially the mature form of human albumin as shown in FIG. 14 and SEQ ID NO:18 herein and in FIG. 15 and SEQ ID NO:18 of U.S. Provisional Application Ser. No. 60/355,547 and WO 01/79480 or albumin from other vertebrates or fragments thereof, or analogs or variants of these molecules or fragments thereof.

The human serum albumin protein used in the albumin fusion proteins of the invention contains one or both of the following sets of point mutations with reference to SEQ ID NO:18: Leu-407 to Ala, Leu-408 to Val, Val-409 to Ala, and Arg-410 to Ala; or Arg-410 to Ala, Lys-413 to Gln, and Lys-414 to Gln (see, e.g., International Publication No. WO95/23857, hereby incorporated in its entirety by reference herein). In other embodiments, albumin fusion proteins of the invention that contain one or both of above-described sets of point mutations have improved stability/resistance to yeast Yap3p proteolytic cleavage, allowing increased production of recombinant albumin fusion proteins expressed in yeast host cells.

As used herein, a portion of albumin sufficient to prolong or extend the in vivo half-life, therapeutic activity, or shelf-life of the Therapeutic protein refers to a portion of albumin sufficient in length or structure to stabilize, prolong or extend the in vivo half-life, therapeutic activity or shelf-life of the Therapeutic protein portion of the albumin fusion protein compared to the in vivo half-life, therapeutic activity, or shelf-life of the Therapeutic protein in the non-fusion state. The albumin portion of the albumin fusion proteins may comprise the full length of the HA sequence as described above, or may include one or more fragments thereof that are capable of stabilizing or prolonging the therapeutic activity. Such fragments may be of 10 or more amino acids in length or may include about 15, 20, 25, 30, 50, or more contiguous amino acids from the HA sequence or may include part or all of specific domains of HA.

The albumin portion of the albumin fusion proteins of the invention may be a variant of normal HA. The Therapeutic protein portion of the albumin fusion proteins of the invention may also be variants of the Therapeutic proteins as described herein. The term “variants” includes insertions, deletions and substitutions, either conservative or non conservative, where such changes do not substantially alter one or more of the oncotic, useful ligand-binding and non-immunogenic properties of albumin, or the active site, or active domain which confers the therapeutic activities of the Therapeutic proteins.

In particular, the albumin fusion proteins of the invention may include naturally occurring polymorphic variants of human albumin and fragments of human albumin, for example those fragments disclosed in EP 322 094 (namely HA (Pn), where n is 369 to 419). The albumin may be derived from any vertebrate, especially any mammal, for example human, cow, sheep, or pig. Non-mammalian albumins include, but are not limited to, hen and salmon. The albumin portion of the albumin fusion protein may be from a different animal than the Therapeutic protein portion.

Generally speaking, an HA fragment or variant will be at least 100 amino acids long, optionally at least 150 amino acids long. The HA variant may consist of or alternatively comprise at least one whole domain of HA, for example domains 1 (amino acids 1-194 of SEQ ID NO:18), 2 (amino acids 195-387 of SEQ ID NO:18), 3 (amino acids 388-585 of SEQ ID NO:18), 1+2 (1-387 of SEQ ID NO:18), 2+3 (195-585 of SEQ ID NO:18) or 1+3 (amino acids 1-194 of SEQ ID NO:18+amino acids 388-585 of SEQ ID NO: 18). Each domain is itself made up of two homologous subdomains namely 1-105, 120-194, 195-291, 316-387, 388-491 and 512-585, with flexible inter-subdomain linker regions comprising residues Lys106 to Glu119, Glu292 to Val315 and Glu492 to Ala511.

The albumin portion of an albumin fusion protein of the invention may comprise at least one subdomain or domain of HA or conservative modifications thereof. If the fusion is based on subdomains, some or all of the adjacent linker is may optionally be used to link to the Therapeutic protein moiety.

Albumin Fusion Proteins

The present invention relates generally to albumin fusion proteins and methods of treating, preventing, or ameliorating diseases or disorders. As used herein, “albumin fusion protein” refers to a protein formed by the fusion of at least one molecule of albumin (or a fragment or variant thereof) to at least one molecule of a Therapeutic protein (or fragment or variant thereof). An albumin fusion protein of the invention comprises at least a fragment or variant of a Therapeutic protein and at least a fragment or variant of human serum albumin, which are associated with one another, such as by genetic fusion (i.e., the albumin fusion protein is generated by translation of a nucleic acid in which a polynucleotide encoding all or a portion of a Therapeutic protein is joined in-frame with a polynucleotide encoding all or a portion of albumin) to one another. The Therapeutic protein and albumin protein, once part of the albumin fusion protein, may be referred to as a “portion”, “region” or “moiety” of the albumin fusion protein.

In one embodiment, the invention provides an albumin fusion protein comprising, or alternatively consisting of, a Therapeutic protein and a serum albumin protein. In other embodiments, the invention provides an albumin fusion protein comprising, or alternatively consisting of, a biologically active and/or therapeutically active fragment of a Therapeutic protein and a serum albumin protein. In other embodiments, the invention provides an albumin fusion protein comprising, or alternatively consisting of, a biologically active and/or therapeutically active variant of a Therapeutic protein and a serum albumin protein. In furtlier embodiments, the serum albumin protein component of the albumin fusion protein is the mature portion of serum albumin.

In further embodiments, the invention provides an albumin fusion protein comprising, or alternatively consisting of, a Therapeutic protein, and a biologically active and/or therapeutically active fragment of serum albumin. In further embodiments, the invention provides an albumin fusion protein comprising, or alternatively consisting of, a Therapeutic protein and a biologically active and/or therapeutically active variant of serum albumin. In some embodiments, the Therapeutic protein portion of the albumin fusion protein is the mature portion of the Therapeutic protein.

In further embodiments, the invention provides an albumin fusion protein comprising, or alternatively consisting of, a biologically active and/or therapeutically active fragment or variant of a Therapeutic protein and a biologically active and/or therapeutically active fragment or valiant of serum albumin. In some embodiments, the invention provides an albumin fusion protein comprising, or alternatively consisting of, the mature portion of a Therapeutic protein and the mature portion of serum albumin.

In one embodiment, the albumin fusion protein comprises HA as the N-terminal portion, and a Therapeutic protein as the C-terminal portion. Alternatively, an albumin fusion protein comprising HA as the C-terminal portion, and a Therapeutic protein as the N-terminal portion may also be used.

In other embodiments, the albumin fusion protein has a Therapeutic protein fused to both the N-terminus and the C-terminus of albumin. In one embodiment, the Therapeutic proteins fused at the N- and C-termini are the same Therapeutic proteins. In another embodiment, the Therapeutic proteins fused at the N- and C-termini are different Therapeutic proteins. In another embodiment, the Therapeutic proteins fused at the N- and C-termini are different Therapeutic proteins which may be used to treat or prevent the same disease, disorder, or condition. In another embodiment, the Therapeutic proteins fused at the N- and C-termini are different Therapeutic proteins which may be used to treat or prevent diseases or disorders which are known in the art to commonly occur in patients simultaneously.

In addition to albumin fusion protein in which the albumin portion is fused N-terminal and/or C-terminal of the Therapeutic protein portion, albumin fusion proteins of the invention may also be produced by inserting the Therapeutic protein or peptide of interest into an internal region of HA. For instance, within the protein sequence of the HA molecule a number of loops or turns exist between the end and beginning of α-helices, which are stabilized by disulphide bonds. The loops, as determined from the crystal structure of HA (PDB identifiers 1AO6, 1BJ5, 1BKE, 1BM0, 1E7E to 1E7I and 1UOR) for the most part extend away from the body of the molecule. These loops are useful for the insertion, or internal fusion, of therapeutically active peptides, particularly those requiring a secondary structure to be functional, or Therapeutic proteins, to essentially generate an albumin molecule with specific biological activity.

Loops in human albumin structure into which peptides or polypeptides may be inserted to generate albumin fusion proteins of the invention include: Val54-Asn61, Thr76-Asp89, Ala92-Glu100, Gln170-Ala176, His247-Glu252, Glu266-Glu277, Glu280-His288, Ala362-Glu368, Lys439-Pro447, Val462-Lys475, Thr478-Pro486, and Lys560-Thr566. In other embodiments, peptides or polypeptides are inserted into the Val54-Asn61, Gln170-Ala176, and/or Lys560-Thr566 loops of mature human albumin (SEQ ID NO:18).

Peptides to be inserted may be derived from either phage display or synthetic peptide libraries screened for specific biological activity or from the active portions of a molecule with the desired function. Additionally, random peptide libraries may be generated within particular loops or by insertions of randomized peptides into particular loops of the HA molecule and in which all possible combinations of amino acids are represented.

Such library(s) could be generated on HA or domain fragments of HA by one of the following methods:

(a) randomized mutation of amino acids within one or more peptide loops of HA or HA domain fragments. Either one, more or all the residues within a loop could be mutated in this manner;

(b) replacement of, or insertion into one or more loops of HA or HA domain fragments (i.e., internal fusion) of a randomized peptide(s) of length Xₙ (where X is an amino acid and n is the number of residues;

(c) N-, C- or N- and C-terminal peptide/protein fusions in addition to (a) and/or (b).

The HA or HA domain fragment may also be made multifunctional by grafting the peptides derived from different screens of different loops against different targets into the same HA or HA domain fragment.

Peptides inserted into a loop of human serum albumin are Therapeutic protein peptides or peptide fragments or peptide variants thereof. For example, peptides inserted into a loop of human serum albumin may include T-20 and/or T-1249 peptide or peptide fragments or peptide variants thereof. More particularly, the invention encompasses albumin fusion proteins which comprise peptide fragments or peptide variants at least 7 at least 8, at least 9, at least 10, at least 11, at least 12, at least 13, at least 14, at least 15, at least 20, at least 25, at least 30, at least 35, or at least 40 amino acids in length inserted into a loop of human serum albumin. The invention also encompasses albumin fusion proteins which comprise peptide fragments or peptide variants at least 7 at least 8, at least 9, at least 10, at least 11, at least 12, at least 13, at least 14, at least 15, at least 20, at least 25, at least 30, at least 35, or at least 40 amino acids fused to the N-terminus of human serum albumin. The invention also encompasses albumin fusion proteins which comprise peptide fragments or peptide variants at least 7 at least 8, at least 9, at least 10, at least 11, at least 12, at least 13, at least 14, at least 15, at least 20, at least 25, at least 30, at least 35, or at least 40 amino acids fused to the C-terminus of human serum albumin.

Generally, the albumin fusion proteins of the invention may have one HA-derived region and one Therapeutic protein-derived region. Multiple regions of each protein, however, may be used to make an albumin fusion protein of the invention. Similarly, more than one Therapeutic protein may be used to make an albumin fusion protein of the invention. For instance, a Therapeutic protein may be fused to both the N- and C-terminal ends of the HA. In such a configuration, the Therapeutic protein portions may be the same or different Therapeutic protein molecules. The structure of bifunctional albumin fusion proteins may be represented as: X-HA-Y or Y-HA-X or X-Y-HA or HA-X-Y or HA-Y-X-HA or HA-X-X-HA or HA Y-Y-HA or HA-X-HA-Y or X-HA-Y-HA or multiple combinations and/or inserting X and/or Y within the HA sequence at any location.

Bi- or multi-functional albumin fusion proteins may be prepared in various ratios depending on function, half-life etc.

Bi- or multi-functional albumin fusion proteins may also be prepared to target the Therapeutic protein portion of a fusion to a target organ or cell type via protein or peptide at the opposite terminus of HA.

As an alternative to the fusion of known therapeutic molecules, the peptides could be obtained by screening libraries constructed as fusions to the N-, C- or N- and C-termini of HA, or domain fragment of HA, of typically 6, 8, 12, 20 or 25 or Xₙ (where X is an amino acid (aa) and n equals the number of residues) randomized amino acids, and in which all possible combinations of amino acids were represented. A particular advantage of this approach is that the peptides may be selected in situ on the HA molecule and the properties of the peptide would therefore be as selected for rather than, potentially, modified as might be the case for a peptide derived by any other method then being attached to FHA.

Additionally, the albumin fusion proteins of the invention may include a linker peptide between the fused portions to provide greater physical separation between the moieties and thus maximize the accessibility of the Therapeutic protein portion, for instance, for binding to its cognate receptor. The linker peptide may consist of amino acids such that it is flexible or more rigid.

Therefore, as described above, the albumin fusion proteins of the invention may have the following formula R2-R1; R1-R2; R2-R1-P2; R2-L-R1-L-R2; R1-L-R2; R2-L-R1; or R1-L-R2-L-R1, wherein R1 is at least one Therapeutic protein, peptide or polypeptide sequence (including fragments or variants thereof), and not necessarily the same Therapeutic protein, L is a linker and R2 is a serum albumin sequence (including fragments or variants thereof). Exemplary linkers include (GGGGS)_{N} (SEQ ID NO:8) or (GGGS)ₜ (SEQ ID NO:9) or (GGS)_{N}, wherein N is an integer greater than or equal to 1 and wherein G represents glycine and S represents serine. When R1 is two or more Therapeutic proteins, peptides or polypeptide sequence, these sequences may optionally be connected by a linker.

In further embodiments, albumin fusion proteins of the invention comprising a Therapeutic protein have extended shelf-life or in vivo half-life or therapeutic activity compared to the shelf-life or in vivo half-life or therapeutic activity of the same Therapeutic protein when not fused to albumin. Shelf-life typically refers to the time period over which the therapeutic activity of a Therapeutic protein in solution or in some other storage formulation, is stable without undue loss of therapeutic activity. Many of the Therapeutic proteins are highly labile in their unfused state. As described below, the typical shelf-life of these Therapeutic proteins is markedly prolonged upon incorporation into the albumin fusion protein of the invention.

Albumin fusion proteins of the invention with “prolonged” or “extended” shelf-life exhibit greater therapeutic activity relative to a standard that has been subjected to the same storage and handling conditions. The standard may be the unfused full-length Therapeutic protein. When the Therapeutic protein portion of the albumin fusion protein is an analog, a variant, or is otherwise altered or does not include the complete sequence for that protein, the prolongation of therapeutic activity may alternatively be compared to the unfused equivalent of that analog, variant, altered peptide or incomplete sequence. As an example, an albumin fusion protein of the invention may retain greater than about 100% of the therapeutic activity, or greater than about 105%, 110%, 120%, 130%, 150% or 200% of the therapeutic activity of a standard when subjected to the same storage and handling conditions as the standard when compared at a given time point. However, it is noted that the therapeutic activity depends on the Therapeutic protein's stability, and may be below 100%.

Shelf-life may also be assessed in terms of therapeutic activity remaining after storage, normalized to therapeutic activity when storage began. Albumin fusion proteins of the invention with prolonged or extended shelf-life as exhibited by prolonged or extended therapeutic activity may retain greater than about 50% of the therapeutic activity, about 60%, 70%, 80%, or 90% or more of the therapeutic activity of the equivalent unfused Therapeutic protein when subjected to the same conditions.

Therapeutic Proteins

As stated above, an albumin fusion protein of the invention comprises at least a fragment or variant of a Therapeutic protein and at least a fragment or variant of human serum albumin, which are associated with one another by genetic fusion.

As used herein, “Therapeutic protein” refers to a HIV fusion inhibiting peptide, (such as T-20, T-1249, 5-Helix or cyanovirin-N), or fragments or variants thereof, having one or more therapeutic and/or biological activities. Thus an albumin fusion protein of the invention may contain at least a fragment or variant of a Therapeutic protein. Additionally, the term “Therapeutic protein” may refer to the endogenous or naturally occurring correlate of a Therapeutic protein. Variants include mutants, analogs, and mimetics, as well as homologs, including the endogenous or naturally occurring correlates of a Therapeutic protein.

By a polypeptide displaying a “therapeutic activity” or a protein that is “therapeutically active” is meant a polypeptide that possesses one or more known biological and/or therapeutic activities associated with a Therapeutic protein such as one or more of the Therapeutic proteins described herein or otherwise known in the art. As a non-limiting example, a “Therapeutic protein” is a protein that is useful to treat, prevent or ameliorate a disease, condition or disorder.

As used herein, “therapeutic activity” or “activity” may refer to an activity whose effect is consistent with a desirable therapeutic outcome in humans, or to desired effects in non-human mammals or in other species or organisms. Therapeutic activity may be measured in vivo or in vitro. For example, a desirable effect may be assayed in cell culture. Such in vitro or cell culture assays are commonly available for many Therapeutic proteins as described in the art.

Examples of useful assays include, but are not limited to, those described in the references and publications in Table 1 (such as U.S. Pat. No. 6,133,418, at column 12, lines 20-58), specifically incorporated by reference herein, and those described in Examples 8 and 11 herein. The antiviral activity exhibited by the fusion proteins of the invention may be measured, for example, by easily performed in vitro assays, such as those described below, which can test the fusion proteins' ability to inhibit syncytia formation, or their ability to inhibit infection by cell-free virus. Using these assays, such parameters as the relative antiviral activity of the fusion proteins exhibit against a given strain of virus and/or the strain specific inhibitory activity of the fusion proteins can be determined. A cell-cell fusion assay may be utilized to test the fusion proteins' ability to inhibit HIV-induced syncytia formation in vitro. Such all assay may comprise culturing uninfected CD-4⁺ cells (such as Molt or CEM cells, for example) in the presence of chronically HIV-infected cells and a peptide to be assayed. For each peptide, a range of peptide concentrations may be tested. This range should include a control culture wherein no peptide has been added. Standard conditions for culturing, well known to those of ordinary skill in the art, are used. After incubation for an appropriate period (24 to 72 hours at 37° C., for example) the culture is examined microscopically for the presence of multinucleated giant cells, which are indicative of cell fusion and syncytia formation.

As another example, a reverse transcriptase (RT) assay may be utilized to test the fusion proteins' ability to inhibit infection of CD-4⁺ cells by cell-free HIV. Such an assay may comprise culturing an appropriate concentration (i.e., TCID₅₀) of virus and CD-4⁺ cells in the presence of the fusion proteins to be tested. Culture conditions well known to those in the art are used. As above, a range of fusion protein concentrations may be used, in addition to a control culture wherein no peptide has been added. After incubation for an appropriate period (e.g., 7 days) of culturing, a cell-free supernatant is prepared, using standard procedures, and tested for the present of RT activity as a measure of successful infection. The RT activity may be tested using standard techniques such as those described by, for example, Goff et al. (Goff, S. et al., 1981, J. Virol. 38:239-248) and/or Willey et al. (Willey, R. et al., 1988, J. Virol. 62:139-147). These references are incorporated herein by reference in their entirety.

Therapeutic proteins corresponding to a Therapeutic protein portion of an albumin fusion protein of the invention may be modified by the attachment of one or more oligosaccharide groups. The modification, referred to as glycosylation, can dramatically affect the physical properties of proteins and can be important in protein stability, secretion, and localization. Such modifications are described in detail in U.S. Provisional Application Ser. No. 60/355,547 and WO 01/79480, which are incorporated herein by reference.

Therapeutic proteins corresponding to a Therapeutic protein portion of an albumin fusion protein of the invention, as well as analogs and variants thereof, may be modified so that glycosylation at one or more sites is altered as a result of manipulation(s) of their nucleic acid sequence, by the host cell in which they are expressed, or due to other conditions of their expression. For example, glycosylation isomers may be produced by abolishing or introducing glycosylation sites, e.g., by substitution or deletion of amino acid residues, such as substitution of glutamine for asparagine, or unglycosylated recombinant proteins may be produced by expressing the proteins in host cells that will not glycosylate them, e.g. in E. coli or glycosylation-deficient yeast. Examples of these approaches are described in more detail in U.S. Provisional Application Ser. No. 60/355,547 and WO 01/79480, which are incorporated by reference, and are known in the art.

Table 1 provides a non-exhaustive list of Therapeutic proteins that correspond to a Therapeutic protein portion of an albumin fusion protein of the invention. The “Therapeutic Protein X” column discloses Therapeutic protein molecules followed by parentheses containing scientific and brand names that comprise, or alternatively consist of, that Therapeutic protein molecule or a fragment or variant thereof. “Therapeutic protein X” as used herein may refer either to an individual Therapeutic protein molecule (as defined by the amino acid sequence obtainable from the CAS and Genbank accession numbers), or to the entire group of Therapeutic proteins associated with a given Therapeutic protein molecule disclosed in this column. The information associated with each of these entries are each incorporated by reference in their entireties, particularly with respect to the amino acid sequences described therein. The “PCT/Patent Reference” column provides U.S. Patent numbers, or PCT International Publication Numbers corresponding to patents and/or published patent applications that describe the Therapeutic protein molecule. Each of the patents and/or published patent applications cited in the “PCT/Patent Reference” column are herein incorporated by reference in their entireties. In particular, the amino acid sequences of the specified polypeptide set forth in the sequence listing of each cited “PCT/Patent Reference”, the variants of these amino acid sequences (mutations, fragments, etc.) set forth, for example, in the detailed description of each cited “PCT/Patent Reference”, the therapeutic indications set forth, for example, in the detailed description of each cited “PCT/Patent Reference”, and the activity assays for the specified polypeptide set forth in the detailed description, and more particularly, the examples of each cited “PCT/Patent Reference” are incorporated herein by reference. The “Biological activity” column describes Biological activities associated with the Therapeutic protein molecule. Each of the references cited in the “Relevant Information” column are herein incorporated by reference in their entireties, particularly with respect to the description of the respective activity assay described in the reference (see Methods section, for example) for assaying the corresponding biological activity. The “Preferred Indication Y” column describes disease, disorders, and/or conditions that may be treated, prevented, diagnosed, or ameliorated by Therapeutic protein X or an albumin fusion protein of the invention comprising a Therapeutic protein X portion.

TABLE 1

| Therapeutic | PCT/Patent | | | Preferred |
|---|---|---|---|---|
| Protein X | Reference | Biological Activity | Relevant Publications | Indication Y |
| HIV- | T-20 | These peptides bind to | T-20: | Treatment of |
| Inhibitors | U.S. Pat. No. 5,464,933, | the envelope proteins | Wild et al. (1993) Aids Res. and | HIV infection |
| (T-20, T- | U.S. Pat. No. 6,060,065, | of HIV and inhibit | Human Retrovir. 9: 1051-1053; Wild et | |
| 1249, | U.S. Pat. No. 6,068,973, | fusion between HIV | al. (1994) PNAS 91: 9770-9774; Chan | |
| cyanovirin | U.S. Pat. No. 6,133,418, | and the target cell. | et al. (1998) PNAS 95: 15613-15617; | |
| and 5-helix) | WO 94/28920 | Cyanovirin targets | Chan et al. (1998) Cell 93: 681-684; | |
| | T-20 and T-1249 | binding to gp120; T-20, | Church et al. (2002) The Ped. Infect. | |
| | U.S. Pat. No. 6,258,782, | T-1249 and 5-Helix | Dis. J. 21: 653-659; Cohen et al. | |
| | U.S. Pat. No. 6,348,568, | target binding to gp41. | ((2002) AIDS Patient Care and STDs | |
| | WO 99/59615, | | 16: 327-335; Este et al. (2001) AIDS | |
| | WO 01/03723, | | Reviews 3: 121-132; Hanna et al. | |
| | WO 01/37896 | | (2002) AIDS 16: 1603-1608; Kilby et | |
| | cyanovirin: | | al. (1998) Nature Med. 4: 1302-1307; | |
| | U.S. Pat. No. 5,821,081, | | Kilby et al. (2002) AIDS Res. and | |
| | U.S. Pat. No. 5,843,882 | | Human Retrovir. 18: 685-693; Lalezari | |
| | Others* | | et al. (2001) 8^{th} Conf. Retrovir. Opp. | |
| | U.S. Pat. No. 5,656,480, | | Inf. Abs. LBS; Rimsky et al. (1998) | |
| | EP0652895 | | Nature Med. 4: 1302-1307; Wei et al. | |
| | *Note that other | | (2002) Antimicro. Agents Chemother. | |
| | HIV fusion | | 46: 1896-1905; | |
| | inhibitors are | | http://199.105.91.6/treatment/Drug/ID14l.ASP; | |
| | also described in | | http://www.thebody.com/gmhc/issues/apr01/T-20.html | |
| | many of the | | T-20 & T-1249 | |
| | other PCT patent | | D'Souza et al. (2000) JAMA 284: 215-222; | |
| | references listed | | Greenberg (2002) Antiviral Ther. | |
| | above | | 7: S106-S107 | |
| | | | 5-Helix: | |
| | | | Root and Kim (2001) Science 291: 884-888; | |
| | | | Hanna et al. (2002) AIDS | |
| | | | 16: 1603-1608 | |
| | | | cyanovirin: | |
| | | | Boyd et al. (1997) Antimicrob. Agents | |
| | | | and Chemotherapy 41: 1521-1530; | |
| | | | Gustafson et al. (1997) BBRC | |
| | | | 238: 223-228; D'Souza et al. (2000) | |
| | | | JAMA 284: 215-222 | |
| | | | Other: | |
| | | | Wild et al. (1992) PNAS 89: 10537-10541; | |
| | | | D'Souza et al. (2000) JAMA | |
| | | | 284: 215-222; Eckert et al. (2001) | |
| | | | PNAS 98: 11187-11192; Eckert et al. | |
| | | | (1999) Cell 99: 103-115; Qureshi et al. | |
| | | | (1990) AIDS 4: 553-558; Wild et al. | |
| | | | (1992) PNAS 89: 10537-10541 | |

In various embodiments, the albumin fusion proteins of the invention are capable of a therapeutic activity and/or biologic activity corresponding to the therapeutic activity and/or biologic activity of the Therapeutic protein corresponding to the Therapeutic protein portion of the albumin fusion protein listed in the corresponding row of Table 1. (See, e.g., the “Biological Activity” and “Therapeutic Protein X” columns of Table 1.) In further embodiments, the therapeutically active protein portions of the albumin fusion proteins of the invention are fragments or variants of the reference sequence and are capable of the therapeutic activity and/or biologic activity of the corresponding Therapeutic protein disclosed in “Biological Activity” column of Table 1.

### Polypeptide and Polynucleotide Fragments and Variants

Fragments

The present invention is further directed to fragments of the Therapeutic proteins described in Table 1, albumin proteins, and/or albumin fusion proteins of the invention.

Even if deletion of one or more amino acids from the N-terminus of a protein results in modification or loss of one or more biological functions of the Therapeutic protein, albumin protein, and/or albumin fusion protein, other Therapeutic activities and/or functional activities (e.g., biological activities, ability to multimerize, ability to bind a ligand) may still be retained. For example, the ability of polypeptides with N-terminal deletions to induce and/or bind to antibodies which recognize the complete or mature forms of the polypeptides generally will be retained when less than the majority of the residues of the complete polypeptide are removed from the N-terminus. Whether a particular polypeptide lacking N-terminal residues of a complete polypeptide retains such immunologic activities can readily be determined by routine methods described herein and otherwise known in the art. It is not unlikely that a mutein with a large number of deleted N-terminal amino acid residues may retain some biological or immunogenic activities. In fact, peptides composed of as few as six amino acid residues may often evoke an immune response.

Accordingly, fragments of a Therapeutic protein corresponding to a Therapeutic protein portion of an albumin fusion protein of the invention, include the full length protein as well as polypeptides having one or more residues deleted from the amino terminus of the amino acid sequence of the reference polypeptide (e.g., a Therapeutic protein as disclosed in Table 1). Polynucleotides encoding these polypeptides are also encompassed by the invention.

In addition, fragments of serum albumin polypeptides corresponding to an albumin protein portion of an albumin fusion protein of the invention, include the full length protein as well as polypeptides having one or more residues deleted from the amino terminus of the amino acid sequence of the reference polypeptide (i.e., serum albumin). Polynucleotides encoding these polypeptides are also encompassed by the invention.

Moreover, fragments of albumin fusion proteins of the invention, include the fall length albumin fusion protein as well as polypeptides having one or more residues deleted from the amino terminus of the albumin fusion protein. Polynucleotides encoding these polypeptides are also encompassed by the invention.

The present invention further provides polypeptides having one or more residues deleted from the carboxy terminus of the amino acid sequence of a Therapeutic protein corresponding to a Therapeutic protein portion of an albumin fusion protein of the invention (e.g., a Therapeutic protein referred to in Table 1). Polynucleotides encoding these polypeptides are also encompassed by the invention.

In addition, the present invention provides polypeptides having one or more residues deleted from the carboxy terminus of the amino acid sequence of an albumin protein corresponding to an albumin protein portion of an albumin fusion protein of the invention (e.g., serum albumin). Polynucleotides encoding these polypeptides are also encompassed by the invention.

Moreover, the present invention provides polypeptides having one or more residues deleted from the carboxy terminus of an albumin fusion protein of the invention. Polynucleotides encoding these polypeptides are also encompassed by the invention.

In addition, any of the above described N- or C-terminal deletions can be combined to produce a N- and C-terminal deleted reference polypeptide (e.g., a Therapeutic protein referred to in Table 1, or serum albumin (e.g., SEQ ID NO:18), or an albumin fusion protein of the invention). The invention also provides polypeptides having one or more amino acids deleted from both the amino and the carboxyl termini. Polynucleotides encoding these polypeptides are also encompassed by the invention.

The present application is also directed to proteins containing polypeptides at least 60%, 80%, 85%, 90%, 95%, 96%, 97%, 98% or 99% identical to a reference polypeptide sequence (e.g., a Therapeutic protein, serum albumin protein or an albumin fusion protein of the invention) set forth herein, or fragments thereof. In some embodiments, the application is directed to proteins comprising polypeptides at least 60%, 80%, 85%, 90%, 95%, 96%, 97%, 98% or 99% identical to reference polypeptides having the amino acid sequence of N- and C-terminal deletions as described above. Polynucleotides encoding these polypeptides are also encompassed by the invention.

Other polypeptide fragments of the invention are fragments comprising, or alternatively, consisting of, an amino acid sequence that displays a Therapeutic activity and/or functional activity (e.g. biological activity) of the polypeptide sequence of the Therapeutic protein or serum albumin protein of which the amino acid sequence is a fragment.

Other polypeptide fragments are biologically active fragments. Biologically active fragments are those exhibiting activity similar, but not necessarily identical, to an activity of the polypeptide of the present invention. The biological activity of the fragments may include an improved desired activity, or a decreased undesirable activity.

Variants

“Variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide.

As used herein, “variant”, refers to a Therapeutic protein portion of an albumin fusion protein of the invention, albumin portion of an albumin fusion protein of the invention, or albumin fusion protein differing in sequence from a Therapeutic protein (e.g. see “therapeutic” column of Table 1), albumin protein, and/or albumin fusion protein of the invention, respectively, but retaining at least one functional and/or therapeutic property thereof (e.g., a therapeutic activity and/or biological activity as disclosed in the “Biological Activity” column of Table 1) as described elsewhere herein or otherwise known in the art. Generally, variants are overall very similar, and, in many regions, identical to the amino acid sequence of the Therapeutic protein corresponding to a Therapeutic protein portion of an albumin fusion protein of the invention, albumin protein corresponding to an albumin protein portion of an albumin fusion protein of the invention, and/or albumin fusion protein of the invention. Nucleic acids encoding these variants are also encompassed by the invention.

The present invention is also directed to proteins which comprise, or alternatively consist of, an amino acid sequence which is at least 60%, 80%, 85%, 90%, 95%, 96%, 97%, 98%, 99% or 100%, identical to, for example, the amino acid sequence of a Therapeutic protein corresponding to a Therapeutic protein portion of an albumin fusion protein of the invention (e.g., an amino acid sequence disclosed in a reference in Table 1, or fragments or variants thereof), albumin proteins (e.g., SEQ ID NO:18 or fragments or variants thereof) corresponding to an albumin protein portion of an albumin fusion protein of the invention, and/or albumin fusion proteins of the invention. Fragments of these polypeptides are also provided (e.g., those fragments described herein). Further polypeptides encompassed by the invention are polypeptides encoded by polynucleotides which hybridize to the complement of a nucleic acid molecule encoding an amino acid sequence of the invention under stringent hybridization conditions (e.g., hybridization to filter bound DNA in 6× Sodium chloride/Sodium citrate (SSC) at about 45 degrees Celsius, followed by one or more washes in 0.2×SSC, 0.1% SDS at about 50-65 degrees Celsius), under highly stringent conditions (e.g., hybridization to filter bound DNA in 6× sodium chloride/Sodium citrate (SSC) at about 45 degrees Celsius, followed by one or more washes in 0.1×SSC, 0.2% SDS at about 68 degrees Celsius), or under other stringent hybridization conditions which are known to those of skill in the art (see, for example, Ausubel, F. M. et al., eds., 1989 Current protocol in Molecular Biology, Green publishing associates, Inc., and John Wiley & Sons Inc., New York, at pages 6.3.1-6.3.6 and 2.10.3). Polynucleotides encoding these polypeptides are also encompassed by the invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 60%, 80%, 85%, 90%, 95%, 96%, 97%, 98% or 99% identical to, for instance, the amino acid sequence of an albumin fusion protein of the invention or a fragment thereof (such as the Therapeutic protein portion of the albumin fusion protein or the albumin portion of the albumin fusion protein), can be determined conventionally using known computer programs. Such programs and methods of using them are described, e.g., in U.S. Provisional Application Ser. No. 60/355,547 and WO 01/79480 (pp. 41-43), which are incorporated by reference herein, and are well known in the art.

The polynucleotide variants of the invention may contain alterations in the coding regions, non-coding regions, or both. Polynucleotide variants include those containing alterations which produce silent substitutions, additions, or deletions, but do not alter the properties or activities of the encoded polypeptide. Such nucleotide variants may be produced by silent substitutions due to the degeneracy of the genetic code. Polypeptide variants include those in which less than 50, less than 40, less than 30, less than 20, less than 10, or 5-50, 5-25, 5-10, 1-5, or 1-2 amino acids are substituted, deleted, or added in any combination. Polynucleotide variants can be produced for a variety of reasons, e.g., to optimize codon expression for a particular host (change codons in the human mRNA to those preferred by a microbial host, such as, yeast or E. coli).

In another embodiment, a polynucleotide encoding an albumin portion of an albumin fusion protein of the invention is optimized for expression in yeast or mammalian cells. In a further embodiment, a polynucleotide encoding a Therapeutic protein portion of an albumin fusion protein of the invention is optimized for expression in yeast or mammalian cells. In a still further embodiment, a polynucleotide encoding an albumin fusion protein of the invention is optimized for expression in yeast or mammalian cells.

In an alternative embodiment, a codon optimized polynucleotide encoding a Therapeutic protein portion of an albumin fusion protein of the invention does not hybridize to the wild type polynucleotide encoding the Therapeutic protein under stringent hybridization conditions as described herein. In a further embodiment, a codon optimized polynucleotide encoding an albumin portion of an albumin fusion protein of the invention does not hybridize to the wild type polynucleotide encoding the albumin protein under stringent hybridization conditions as described herein. In another embodiment, a codon optimized polynucleotide encoding an albumin fusion protein of the invention does not hybridize to the wild type polynucleotide encoding the Therapeutic protein portion or the albumin protein portion under stringent hybridization conditions as described herein.

In an additional embodiment, polynucleotides encoding a Therapeutic protein portion of an albumin fusion protein of the invention do not comprise, or alternatively consist of, the naturally occurring sequence of that Therapeutic protein. In a further embodiment, polynucleotides encoding an albumin protein portion of an albumin fusion protein of the invention do not comprise, or alternatively consist of, the naturally occurring sequence of albumin protein. In an alternative embodiment, polynucleotides encoding an albumin fusion protein of the invention do not comprise, or alternatively consist of, the naturally occurring sequence of a Therapeutic protein portion or the albumin protein portion.

In an additional embodiment, the Therapeutic protein may be selected from a random peptide library by biopanning, as there will be no naturally occurring wild type polynucleotide.

Naturally occurring variants are called “allelic variants,” and refer to one of several alternate forms of a gene occupying a given locus on a chromosome of an organism. (Genes II, Lewin, B., ed., John Wiley & Sons, New York (1985)). These allelic variants can vary at either the polynucleotide and/or polypeptide level and are included in the present invention. Alternatively, non-naturally occurring variants may be produced by mutagenesis techniques or by direct synthesis.

Using known methods of protein engineering and recombinant DNA technology, variants may be generated to improve or alter the characteristics of the polypeptides of the present invention. For instance, one or more amino acids may be deleted from the N-terminus or C-terminus of the polypeptide of the present invention without substantial loss of biological function. See, e.g., Ron et al., J. Biol. Chem. 268: 2984-2988 (199D) (KGF variants) and Dobeli et al., J. Biotechnology 7:199-216 (1988) (interferon gamma variants).

Moreover, ample evidence demonstrates that variants often retain a biological activity similar to that of the naturally occurring protein (eg Gayle and coworkers (J. Biol. Chem. 268:22105-22111 (1993) (IL-1a variants)). Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes polypeptide variants which have a functional activity (e.g., biological activity and/or therapeutic activity). In further embodiments the invention provides variants of albumin fusion proteins that have a functional activity (e.g., biological activity and/or therapeutic activity, such as that disclosed in the “Biological Activity” column in Table 1) that corresponds to one or more biological and/or therapeutic activities of the Therapeutic protein corresponding to the Therapeutic protein portion of the albumin fusion protein. Such variants include deletions, insertions, inversions, repeats, and substitutions selected according to general rules known in the art so as have little effect on activity.

In other embodiments, the variants of the invention have conservative substitutions. By “conservative substitutions” is intended swaps within groups such as replacement of the aliphatic or hydrophobic amino acids Ala, Val, Leu and Ile; replacement of the hydroxyl residues Ser and Thr; replacement of the acidic residues Asp and Glu; replacement of the amide residues Asn and Ghn, replacement of the basic residues Lys, Arg, and His; replacement of the aromatic residues Phe, Tyr, and Trp, and replacement of the small-sized amino acids Ala, Ser, Thr, Met, and Gly.

Guidance concerning how to make phenotypically silent amino acid substitutions is provided, for example, in Bowie et al., “Deciphering the Message in Protein Sequences: Tolerance to Amino Acid Substitutions,” Science 247:1306-1310 (1990), wherein the authors indicate that there are two main strategies for studying the tolerance of an amino acid sequence to change.

As the authors state, proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Besides conservative amino acid substitution, variants of the present invention include (i) polypeptides containing substitutions of one or more of the non-conserved amino acid residues, where the substituted amino acid residues may or may not be one encoded by the genetic code, or (ii) polypeptides containing substitutions of one or more of the amino acid residues having a substituent group, or (iii) polypeptides which have been fused with or chemically conjugated to another compound, such as a compound to increase the stability and/or solubility of the polypeptide (for example, polyethylene glycol), (iv) polypeptide containing additional amino acids, such as, for example, an IgG Fc fusion region peptide. Such variant polypeptides are deemed to be within the scope of those skilled in the art from the teachings herein.

For example, polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. See Pinckard et al., Clin. Exp. Immunol. 2:331-340 (1967); Robbins et al., Diabetes 36: 938-845 (1987); Cleland et al., Crit. Rev. Therapeutic Drug Carrier Systems 10:307-377 (1993).

In specific embodiments, the polypeptides of the invention comprise, or alternatively, consist of, fragments or variants of the amino acid sequence of a Therapeutic protein described herein and/or human serum albumin, and/or albumin fusion protein of the invention, wherein the fragments or variants have 1-5, 5-10, 5-25, 5-50, 10-50 or 50-150, amino acid residue additions, substitutions, and/or deletions when compared to the reference amino acid sequence. In certain embodiments, the amino acid substitutions are conservative. Nucleic acids encoding these polypeptides are also encompassed by the invention.

The polypeptide of the present invention can be composed of amino acids joined to each other by peptide bonds or modified peptide bonds, i.e., peptide isosteres, and may contain amino acids other than the 20 gene-encoded amino acids. The polypeptides may be modified by either natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination.

Furthermore, chemical entities may be covalently attached to the albumin fusion proteins to enhance or modulate a specific functional or biological activity such as by methods disclosed in Current Opinions in Biotechnology, 10:324 (1999).

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The albumin fusion proteins may also be modified with, e.g., but not limited to, a chemotherapeutic agent, such as a drug, and/or a detectable label, such as an enzymatic, fluorescent, isotopic and/or affinity label to allow for detection and isolation of the protein. Examples of such modifications are given, e.g., in U.S. Provisional Application Ser. No. 60/355,547 and in WO 01/79480 (pp. 105-106), which are incorporated by reference herein, and are well known in the art.

Functional Activity

“A polypeptide having functional activity” refers to a polypeptide capable of displaying one or more known functional activities associated with the full-length, pro-protein, and/or mature form of a Therapeutic protein. Such functional activities include, but are not limited to, biological activity, antigenicity [ability to bind (or compete with a polypeptide for binding) to an anti-polypeptide antibody], immunogenicity (ability to generate antibody which binds to a specific polypeptide of the invention), ability to form multimers with polypeptides of the invention, and ability to bind to a receptor or ligand for a polypeptide.

“A polypeptide having biological activity” refers to a polypeptide exhibiting activity similar to, but not necessarily identical to, an activity of a Therapeutic protein of the present invention, including mature forms, as measured in a particular biological assay, with or without dose dependency. In the case where dose dependency does exist, it need not be identical to that of the polypeptide, but rather substantially similar to the dose-dependence in a given activity as compared to the polypeptide of the present invention.

In other embodiments, an albumin fusion protein of the invention has at least one biological and/or therapeutic activity associated with the Therapeutic protein (or fragment or variant thereof) when it is not fused to albumin.

The albumin fusion proteins of the invention can be assayed for functional activity (e.g., biological activity) using or routinely modifying assays known in the art, as well as assays described herein. Specifically, albumin fusion proteins may be assayed for functional activity (e.g., biological activity or therapeutic activity) using the assay referenced in the “Relevant Publications” column of Table 1. Additionally, one of skill in the art may routinely assay fragments of a Therapeutic protein corresponding to a Therapeutic protein portion of an albumin fusion protein of the invention, for activity using assays referenced in its corresponding row of Table 1. Further, one of skill in the art may routinely assay fragments of an albumin protein corresponding to an albumin protein portion of an albumin fusion protein of the invention, for activity using assays known in the art and/or as described in the Examples section in U.S. Provisional Application Ser. No. 60/355,547 and WO 01/79480.

In addition, assays described herein (see Examples and Table 1) and otherwise known in the art may routinely be applied to measure the ability of albumin fusion proteins of the present invention and fragments, variants and derivatives thereof to elicit biological activity and/or Therapeutic activity (either in vitro or in vivo) related to either the Therapeutic protein portion and/or albumin portion of the albumin fusion protein of the present invention. Other methods will be known to the skilled artisan and are within the scope of the invention.

Expression of Fusion Proteins

The albumin fusion proteins of the invention may be produced as recombinant molecules by secretion from yeast, a microorganism such as a bacterium, or a human or animal cell line. Optionally, the polypeptide is secreted from the host cells.

For expression of the albumin fusion proteins exemplified herein, yeast strains disrupted of the HSP150 gene as exemplified in WO 95/33833, or yeast strains disrupted of the PMT1 gene as exemplified in WO 00/44772 [rHA process] (serving to reduce/eliminate O-linked glycosylation of the albumin fusions), or yeast strains disrupted of the YAP3 gene as exemplified in WO 95/23857 were successfully used, in combination with the yeast PRB1 promoter, the HSA/MFα-1 fusion leader sequence exemplified in WO 90/01063, the yeast ADH1 terminator, the LEU2 selection marker and the disintegration vector pSAC35 exemplied in U.S. Pat. No. 5,637,504.

Other yeast strains, promoters, leader sequences, terminators, markers and vectors which are expected to be useful in the invention are described in U.S. Provisional Application Ser. No. 60/355,547 and in WO 01/74980 (pp. 94-99), which are incorporated herein by reference, and are well known in the art.

The present invention also includes a cell, optionally a yeast cell transformed to express an albumin fusion protein of the invention. In addition to the transformed host cells themselves, the present invention also contemplates a culture of those cells, optionally a monoclonal (clonally homogeneous) culture, or a culture derived from a monoclonal culture, in a nutrient medium. If the polypeptide is secreted, the medium will contain the polypeptide, with the cells, or without the cells if they have been filtered or centrifuged away. Many expression systems are known and may be used, including bacteria (for example E. coli and Bacillus subtilis), yeasts (for example Saccharomyces cerevisiae, Kluyveromyces lactis and Pichia pastoris), filamentous fungi (for example Aspergillus), plant cells, animal cells and insect cells.

The desired protein is produced in conventional ways, for example from a coding sequence inserted in the host chromosome or on a free plasmid. The yeasts are transformed with a coding sequence for the desired protein in any of the usual ways, for example electroporation. Methods for transformation of yeast by electroporation are disclosed in Becker & Guarente (1990) Methods Enzymol. 194, 182.

Successfully transformed cells, i.e., cells that contain a DNA construct of the present invention, can be identified by well known techniques. For example, cells resulting from the introduction of an expression construct can be grown to produce the desired polypeptide. Cells can be harvested and lysed and their DNA content examined for the presence of the DNA using a method such as that described by Southern (1975) a Mol. Biol. 98, 503 or Berent et al. (1985) Biotech. 3, 208. Alternatively, the presence of the protein in the supernatant can be detected using antibodies.

Useful yeast plasmid vectors include pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, TRP1, LEU2 and URA3. Plasmids pRS413-416 are Yeast Centromere plasmids (YCps).

Vectors for making albumin fusion proteins for expression in yeast include pPPC0005, pScCHSA, pScNHSA, and pC4:HSA which were deposited on Apr. 11, 2001 at the American Type Culture Collection, 10801 University Boulevard, Manassas, Va. 20110-2209 and which are described in Provisional Application Ser. No. 60/355,547 and WO 01/79480, which are incorporated by reference herein.

Another vector which is expected to be useful for expressing an albumin fusion protein in yeast is the pSAC35 vector which is described in Sleep et al., BioTechnology 8:42 (1990), which is hereby incorporated by reference in its entirety. The plasmid pSAC35 is of the disintegration class of vector described in U.S. Pat. No. 5,637,504.

A variety of methods have been developed to operably link DNA to vectors via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase I, enzymes that remove protruding, γ-single-stranded termini with their 3′ 5′-exonucleolytic activities, and fill in recessed 3′-ends with their polymerizing activities. The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to all expression vector that has been cleaved with an enzyme that produces termini compatible with those of the DNA segment.

Synthetic linkers containing a variety of restriction endonuclease sites are commercially available from a number of commercial sources.

A desirable way to modify the DNA in accordance with the invention, if, for example, HA variants are to be prepared, is to use the polymerase chain reaction as disclosed by Saiki et al. (1988) Science 239, 487-491. In this method the DNA to be enzymatically amplified is flanked by two specific oligonucleotide primers which themselves become incorporated into the amplified DNA. The specific primers may contain restriction endonuclease recognition sites which can be used for cloning into expression vectors using methods known in the art.

Exemplary genera of yeast contemplated to be useful in the practice of the present invention as hosts for expressing the albumin fusion proteins are Pichia (formerly classified as Hansenula), Saccharomyces, Kluyveromyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopsis, and the like. Genera include those selected from the group consisting of Saccharomyces, Schizosaccharomyces, Kluyveromyces, Pichia and Torulaspora. Examples of Saccharomyces spp. are S. cerevisiae, S. italicus and S. rouxii. Examples of other species, and methods of transforming them, are described in U.S. Provisional Application Ser. No. 60/355,547 and W 01/79480 (pp. 97-98), which are incorporated herein by reference.

Methods for the transformation of S. cerevisiae are taught generally in EP 251 744, EP 258 067 and WO 90/01063, all of which are incorporated herein by reference.

Suitable promoters for S. cerevisiae include those associated with the PGKI gene, GAL1 or GAL10 genes, CYCI, PHO5, TRPI, ADHI, ADH2, the genes for glyceraldehyde-3-phosphate dehydrogenase, hexokinase, pyruvate decarboxylase, phosphofructokinase, triose phosphate isomerase, phosphoglucose isomerase, glucokinase, alpha-mating factor pheromone, [a mating factor pheromone], the PRBI promoter, the GUT2 promoter, the GPDI promoter, and hybrid promoters involving hybrids of parts of 5′ regulatory regions with parts of 5′ regulatory regions of other promoters or with upstream activation sites (e.g. the promoter of EP-A-258 067).

Convenient regulatable promoters for use in Schizosaccharomyces pombe are the thiamine-repressible promoter from the nmt gene as described by Maundrell (1990) J. Biol. Chem. 265, 10857-10864 and the glucose repressible jbpl gene promoter as described by Hoffman & Winston (1990) Genetics 124, 807-816.

Methods of transforming Pichia for expression of foreign genes are taught in, for example, Cregg et al. (1993), and various Phillips patents (e.g. U.S. Pat. No. 4,857,467, incorporated herein by reference), and Pichia expression kits are commercially available from Invitrogen BV, Leek, Netherlands, and Invitrogen Corp., San Diego, Calif. Suitable promoters include AOXI and AOX2. Gleeson et al (1986) J. Gen. Microbiol. 132, 3459-3465 include information on Hansenula vectors and transformation, suitable promoters being MOX1 and FMD1; whilst EP 361 991, Fleer et al. (1991) and other-publications from Rhone-Poulenc Rorer teach how to express foreign proteins in Kluyveromyces spp.

The transcription termination signal may be the 3′ flanking sequence of a eukaryotic gene which contains proper signals for transcription termination and polyadenylation. Suitable 3′ flanking sequences may, for example, be those of the gene naturally linked to the expression control sequence used, i.e. may correspond to the promoter. Alternatively, they may be different in which case the termination signal of the S. cerevisiae ADHI gene is optionally used.

The desired albumin fusion protein may be initially expressed with a secretion leader sequence, which may be any leader effective in the yeast chosen. Leaders useful in S. cerevisiae include that from the mating factor α polypeptide (MF α-1) and the hybrid leaders of EP-A-387 319. Such leaders (or signals) are cleaved by the yeast before the mature albumin is released into the surrounding medium. Further such leaders include those of S. cerevisiae invertase (SUC2) disclosed in JP 62-096086 (granted as 911036516), acid phosphatase (PH05), the pre-sequence of MFα-1, 0 glucanase (BGL2) and killer toxin; S. diastaticus glucoamylase II; S. carlsbergensis α-galactosidase (MEL1); K. lactis killer toxin; and Candida glucoamylase.

Additional Methods of Recombinant and Synthetic Production of Albumin Fusion Proteins

The present invention includes polynucleotides encoding albumin fusion proteins of this invention, as well as vectors, host cells and organisms containing these polynucleotides. The present invention also includes methods of producing albumin fusion proteins of the invention by synthetic and recombinant techniques. The polynucleotides, vectors, host cells, and organisms may be isolated and purified by methods known in the art

A vector useful in the invention may be, for example, a phage, plasmid, cosmid, mini-chromosome, viral or retroviral vector.

The vectors which can be utilized to clone and/or express polynucleotides of the invention are vectors which are capable of replicating and/or expressing the polynucleotides in the host cell in which the polynucleotides are desired to be replicated and/or expressed. In general, the polynucleotides and/or vectors can be utilized in any cell, either eukaryotic or prokaryotic, including mammalian cells (e.g., human (e.g., HeLa), monkey (e.g., Cos), rabbit (e.g., rabbit reticulocytes), rat, hamster (e.g., CHO, NSO and baby hamster kidney cells) or mouse cells (e.g., L cells), plant cells, yeast cells, insect cells or bacterial cells (e.g., E. coli). See, e.g., F. Ausubel et al., Current Protocols in Molecular Biology, Greene Publishing Associates and Wiley-Interscience (1992) and Sambrook et al. (1989) for examples of appropriate vectors for various types of host cells. Note, however, that when a retroviral vector that is replication defective is used, viral propagation generally will occur only in complementing host cells.

The host cells containing these polynucleotides can be used to express large amounts of the protein useful in, for example, pharmaceuticals, diagnostic reagents, vaccines and therapeutics. The protein may be isolated and purified by methods known in the art or described herein.

The polynucleotides encoding albumin fusion proteins of the invention may be joined to a vector containing a selectable marker for propagation in a host. Generally, a plasmid vector may be introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter compatible with the host cell in which the polynucleotide is to be expressed. The promoter may be a strong promoter and/or an inducible promoter. Examples of promoters include the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs may include a translation initiating codon at the beginning and a termination codon (UAA, UGA or UAG) appropriately positioned at the end of the polypeptide to be translated.

As indicated, the expression vectors may include at least one selectable marker. Such markers include dihydrofolate reductase, G418, glutamine synthase, or neomycin resistance for eukaryotic cell culture, and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, NSO, 293, and Bowes melanoma cells; and plant cells. Appropriate culture mediums and conditions for the above-described host cells are known in the art.

In one embodiment, polynucleotides encoding an albumin fusion protein of the invention may be fused to signal sequences which will direct the localization of a protein of the invention to particular compartments of a prokaryotic or eukaryotic cell and/or direct the secretion of a protein of the invention from a prokaryotic or eukaryotic cell. For example, in E. coli, one may wish to direct the expression of the protein to the periplasmic space. Examples of signal sequences or proteins (or fragments thereof) to, which the albumin fusion proteins of the invention may be fused in order to direct the expression of the polypeptide to the periplasmic space of bacteria include, but are not limited to, the pelB signal sequence, the maltose binding protein (MBP) signal sequence, MBP, the ompA signal sequence, the signal sequence of the periplasmic E. coli heat-labile enterotoxin B-subunit, and the signal sequence of alkaline phosphatase. Several vectors are commercially available for the construction of fusion proteins which will direct the localization of a protein, such as the pMAL series of vectors (particularly the pMAL-p series) available from New England Biolabs. In a specific embodiment, polynucleotides albumin fusion proteins of the invention may be fused to the pelB pectate lyase signal sequence to increase the efficiency of expression and purification of such polypeptides in Gram-negative bacteria. See, U.S. Pat. Nos. 5,576,195 and 5,846,818, the contents of which are herein incorporated by reference in their entireties.

Examples of signal peptides that may be fused to an albumin fusion protein of the invention in order to direct its secretion in mammalian cells include, but are not limited to, the MPIF-1 signal sequence (e.g., amino acids 1-21 of GenBank Accession number AAB51134), the stanniocalcin signal sequence (MLQNSAVLLLLVISASA, SEQ ID NO:10) and a consensus signal sequence (NPTWAWWLFLVLLLALWAPARG, SEQ ID NO:11). A suitable signal sequence that may be used in conjunction with baculoviral expression systems is the gp67 signal sequence (e.g., amino acids 1-19 of GenBank Accession Number AAA72759).

Vectors which use glutamine synthase (GS) or DHFR as the selectable markers can be amplified in the presence of the drugs methionine sulphoximine or methotrexate, respectively. An advantage of glutamine synthase based vectors are the availability of cell lines (e.g., the murine myeloma cell line, NSO) which are glutamine synthase negative. Glutamine synthase expression systems can also function in glutamine synthase expressing cells (e.g., Chinese Hamster Ovary (CHO) cells) by providing additional inhibitor to prevent the functioning of the endogenous gene. A glutamine synthase expression system and components thereof are detailed in PCT publications: WO87/04462; WO86/05807; WO89/01036; WO89/10404; and WO91/06657, which are hereby incorporated in their entireties by reference herein. Additionally, glutamine synthase expression vectors can be obtained from Lonza Biologics, Inc. (Portsmouth, N.H.). Expression and production of monoclonal antibodies using a GS expression system in murine myeloma cells is described in Bebbington et al., Bio/technology 10:169 (1992) and in Biblia and Robinson Biotechnol. Prog. 11:1 (1995) which are herein incorporated by reference.

The present invention also relates to host cells containing vector constructs, such as those described herein, and additionally encompasses host cells containing nucleotide sequences of the invention that are operably associated with one or more heterologous control regions (e.g., promoter and/or enhancer) using techniques known of in the art. The host cell can be a higher eukaryotic cell, such as a mammalian cell (e.g., a human derived cell), or a lower eukaryotic cell, such as a yeast cell, or the host cell can be a prokaryotic cell, such as a bacterial cell. A host strain may be chosen which modulates the expression of the inserted gene sequences, or modifies and processes the gene product in the specific fashion desired. Expression from certain promoters can be elevated in the presence of certain inducers; thus expression of the genetically engineered polypeptide may be controlled. Furthermore, different host cells have characteristics and specific mechanisms for the translational and post-translational processing and modification (e.g., phosphorylation, cleavage) of proteins. Appropriate cell lines can be chosen to ensure the desired modifications and processing of the foreign protein expressed.

Introduction of the nucleic acids and nucleic acid constructs of the invention into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in, fact be expressed by a host cell lacking a recombinant vector.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., the coding sequence corresponding to a Therapeutic protein may be replaced with an albumin fusion protein corresponding to the Therapeutic protein), and/or to include genetic material (e.g., heterologous polynucleotide sequences such as for example, an albumin fusion protein of the invention corresponding to the Therapeutic protein may be included). The genetic material operably associated with the endogenous polynucleotide may activate, alter, and/or amplify endogenous polynucleotides.

In addition, techniques known in the art may be used to operably associate heterologous polynucleotides (e.g., polynucleotides encoding an albumin protein, or a fragment or variant thereof) and/or heterologous control regions (e.g., promoter and/or enhancer) with endogenous polynucleotide sequences encoding a Therapeutic protein via homologous recombination (see, e.g., U.S. Pat. No. 5,641,670, issued Jun. 24, 1997; International Publication Number WO 96/29411; International Publication Number WO 94/12650; Koller et al., Proc. Natl. Acad. Sci. USA 86:8932-8935 (1989); and Zijlstra et al., Nature 342:435-438 (1989), the disclosures of each of which are incorporated by reference in their entireties).

Advantageously, albumin fusion proteins of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, hydrophobic charge interaction chromatography and lectin chromatography. In some embodiments, high performance liquid chromatography (“HPLC”) may be employed for purification.

In preferred some embodiments albumin fusion proteins of the invention are purified using one or more Chromatography methods listed above. In other embodiments, albumin fusion proteins of the invention are purified using one or more of the following Chromatography columns, Q sepharose FF column, SP Sepharose FF column, Q Sepharose High Performance Column, Blue Sepharose FF column, Blue Column, Phenyl Sepharose FF column, DEAE Sepharose FF, or Methyl Column.

Additionally, albumin fusion proteins of the invention may be purified using the process described in International Publication No. WO 00/44772 which is herein incorporated by reference in its entirety. One of skill in the art could easily modify the process described therein for use in the purification of albumin fusion proteins of the invention.

Albumin fusion proteins of the present invention may be recovered from: products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, albumin fusion proteins of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

Albumin fusion proteins of the invention and antibodies that bind a Therapeutic protein or fragments or variants thereof can be fused to marker sequences, such as a peptide to facilitate purification. In one embodiment, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “FLAG” tag.

Further, an albumin fusion protein of the invention may be conjugated to a therapeutic moiety such as a cytotoxin, e.g., a cytostatic or cytocidal agent, a therapeutic agent or a radioactive metal ion, e.g., alpha-emitters such as, for example, 213Bi. Examples of such agents are given in U.S. Provisional Application Ser. No. 60/355,547 and in WO 01/79480 (p. 107), which are incorporated herein by reference.

Albumin fusion proteins may also be attached to solid supports, which are particularly useful for immunoassays or purification of polypeptides that are bound by, that bind to, or associate with albumin fusion proteins of the invention. Such solid supports include, but are not limited to, glass, cellulose, polyacrylamide, nylon, polystyrene, polyvinyl chloride or polypropylene.

Also provided by the invention are chemically modified derivatives of the albumin fusion proteins of the invention which may provide additional advantages such as increased solubility, stability and circulating time of the polypeptide, or decreased immunogenicity (see U.S. Pat. No. 4,179,337). Examples involving the use of polyethylene glycol are given in WO 01/79480 (pp. 109-111), which are incorporated by reference herein.

The presence and quantity of albumin fusion proteins of the invention may be determined using ELISA, a well known immunoassay known in the art.

Uses of the Polypeptides

Each of the polypeptides identified herein can be used in numerous ways. The following description should be considered exemplary and utilizes known techniques.

The albumin fusion proteins of the present invention are useful for treatment, prevention and/or prognosis of various disorders in mammals, preferably humans. Such disorders include, but are not limited to, those described herein under the heading “Biological Activity” in Table 1.

The albumin fusion proteins of the invention may be used as inhibitors of human and non-human retroviral, especially HIV, transmission to uninfected cells. The human retroviruses whose transmission may be inhibited by the peptides of the invention include, but are not limited to all strains of HIV-1 and HIV-2 and the human T-lymphocyte viruses (HTLV-I, II, III). The non-human retroviruses whose transmission may be inhibited by the peptides of the invention include, but are not limited to bovine leukosis virus, feline sarcoma and leukemia viruses, simian sarcoma and leukemia viruses, and sheep progress pneumonia viruses.

Moreover, albumin fusion proteins of the present invention can be used to treat or prevent diseases or conditions. With respect to HIV, the albumin fusion proteins of the invention may be used as a prophylactic or therapeutic in the prevention or treatment of AIDS or other HIV related diseases or disorders.

In addition, the albumin fusion proteins of the invention may be used as a prophylactic measure in previously uninfected individuals after acute exposure to an HIV virus. Examples of such prophylactic use of the peptides may include, but are not limited to, prevention of virus transmission from mother to infant and other settings where the likelihood of HIV transmission exists, such as, for example, accidents in health care settings wherein workers are exposed to HIV-containing blood products. The albumin fusion proteins of the invention in such cases may serve the role of a prophylactic vaccine, wherein the host raises antibodies against the albumin fusion proteins of the invention, which then serve to neutralize HIV viruses by, for example, inhibiting further HIV infection.

Albumin fusion proteins can be used to assay levels of polypeptides in a biological sample. For example, radiolabeled albumin fusion proteins of the invention could be used for imaging of viral nodes in a body. Examples of assays are given, e.g., in U.S. Provisional Application Ser. No. 60/355,547 and WO 0179480 (pp. 112-122), which are incorporated herein by reference, and are well known in the art.

Albumin fusion proteins of the invention can also be used to raise antibodies, which in turn may be used to measure protein expression of the Therapeutic protein, albumin protein, and/or the albumin fusion protein of the invention from a recombinant cell, as a way of assessing transformation of the host cell, or in a biological sample. Moreover, the albumin fusion proteins of the present invention can be used to test the biological activities described herein.

Transgenic Organisms

Transgenic organisms that express the albumin fusion proteins of the invention are also included in the invention. Transgenic organisms are genetically modified organisms into which recombinant, exogenous or cloned genetic material has been transferred. Such genetic material is often referred to as a transgene. The nucleic acid sequence of the transgene may include one or more transcriptional regulatory sequences and other nucleic acid sequences such as introns, that may be necessary for optimal expression and secretion of the encoded protein. The transgene may be designed to direct the expression of the encoded protein in a manner that facilitates its recovery from the organism or from a product produced by the organism, e.g. from the milk, blood, urine, eggs, hair or seeds of the organism. The transgene may consist of nucleic acid sequences derived from the genome of the same species or of a different species than the species of the target animal. The transgene may be integrated either at a locus of a genome where that particular nucleic acid sequence is not otherwise normally found or at the normal locus for the transgene.

The term “germ cell line transgenic organism” refers to a transgenic organism in which the genetic alteration or genetic information was introduced into a germ line cell, thereby conferring the ability of the transgenic organism to transfer the genetic information to offspring. If such offspring in fact possess some or all of that alteration or genetic information, then they too are transgenic organisms. The alteration or genetic information may be foreign to the species of organism to which the recipient belongs, foreign only to the particular individual recipient, or may be genetic information already possessed by the recipient. In the last case, the altered or introduced gene may be expressed differently than the native gene.

A transgenic organism may be a transgenic human, animal or plant. Transgenics can be produced by a variety of different methods including transfection, electroporation, microinjection, gene targeting in embryonic stem cells and recombinant viral and retroviral infection (see, e.g., U.S. Pat. No. 4,736,866; U.S. Pat. No. 5,602,307; Mullins et al. (1993) Hypertension 22(4):630-633; Brenin et al. (1997) Surg. Oncol. 6(2)99-110; Tuan (ed.), Recombinant Gene Expression Protocols, Methods in Molecular Biology No. 62, Humana Press (1997)). The method of introduction of nucleic acid fragments into recombination competent mammalian cells can be by any method which favors co-transformation of multiple nucleic acid molecules. Detailed procedures for producing transgenic animals are readily available to one skilled in the art, including the disclosures in U.S. Pat. No. 5,489,743 and U.S. Pat. No. 5,602,307. Additional information is given in U.S. Provisional Application Ser. No. 60/355,547 and WO 01/79480 (pp. 151-162), which are incorporated by reference herein.

Gene Therapy

Constructs encoding albumin fusion proteins of the invention can be used as a part of a gene therapy protocol to deliver therapeutically effective doses of the albumin fusion protein. One approach for in vivo introduction of nucleic acid into a cell is by use of a viral vector containing nucleic acid, encoding an albumin fusion protein of the invention. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid. The extended plasma half-life of the described albumin fusion proteins might even compensate for a potentially low expression level.

Retrovirus vectors and adeno-associated virus vectors can be used as a recombinant gene delivery system for the transfer of exogenous nucleic acid molecules encoding albumin fusion proteins in vivo. These vectors provide efficient delivery of nucleic acids into cells, and the transferred nucleic acids are stably integrated into the chromosomal DNA of the host. Examples of such vectors, methods of using them, and their advantages, as well as non-viral delivery methods are described in detail in U.S. Provisional Application Ser. No. 60/355,547 and WO 01/79480 (pp. 151-153), which are incorporated by reference herein.

Gene delivery systems for a gene encoding an albumin fusion protein of the invention can be introduced into a patient by any of a number of methods. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression di-e to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by Stereotactic injection (e.g. Chen et al. (1994) PNAS 91: 3054-3057). The pharmaceutical preparation of the gene therapy construct can consist essentially of the gene delivery system in an acceptable diluent, or can comprise a slow release matrix in which the gene delivery vehicle is imbedded. Where the albumin fusion protein can be produced intact from recombinant cells, e.g. retroviral vectors, the pharmaceutical preparation can comprise one or more cells which produce the albumin fusion protein. Additional gene therapy methods are described in U.S. Provisional Application Ser. No. 60/355,547 and in WO 01/79480 (pp. 153-162), which are incorporated herein by reference.

Pharmaceutical or Therapeutic Compositions

The albumin fusion proteins of the invention or formulations thereof may be administered by any conventional method including parenteral (e.g. subcutaneous or intramuscular) injection or intravenous infusion. The treatment may consist of a single dose or a plurality of doses over a period of time. Furthermore, the dose, or plurality of doses, is administered less frequently than for the Therapeutic Protein which is not fused to albumin.

While it is possible for an albumin fusion protein of the invention to be administered alone, it is desirable to present it as a pharmaceutical formulation, together with one or more acceptable carriers. The carrier(s) must be “acceptable” in the sense of being compatible with the albumin fusion protein and not deleterious to the recipients thereof. Typically, the carriers will be water or saline which will be sterile and pyrogen free. Albumin fusion proteins of the invention are particularly well suited to formulation in aqueous carriers such as sterile pyrogen free water, saline or other isotonic solutions because of their extended shelf-life in solution. For instance, pharmaceutical compositions of the invention may be formulated well in advance in aqueous form, for instance, weeks or months or longer time periods before being dispensed.

Formulations containing the albumin fusion protein may be prepared taking into account the extended shelf-life of the albumin fusion protein in aqueous formulations. As discussed above, the shelf-life of many of these Therapeutic proteins are markedly increased or prolonged after fusion to HA.

In instances where aerosol administration is appropriate, the albumin fusion proteins of the invention can be formulated as aerosols using standard procedures. The term “aerosol” includes any gas-borne suspended phase of an albumin fusion protein of the instant invention which is capable of being inhaled into the bronchioles or nasal passages. Specifically, aerosol includes a gas-borne suspension of droplets of an albumin fusion protein of the instant invention, as may be produced in a metered dose inhaler or nebulizer, or in a mist sprayer. Aerosol also includes a dry powder composition of a compound of the instant invention suspended in air or other carrier gas, which may be delivered by insufflation from an inhaler device, for example.

The formulations may conveniently be presented in unit dosage form and may be prepared by any of the methods well known in the art of pharmacy. Such methods include the step of bringing into association the albumin fusion protein with the carrier that constitutes one or more accessory ingredients. In general the formulations are prepared by uniformly and intimately bringing into association the active ingredient with liquid carriers or finely divided solid carriers or both, and then, if necessary, shaping the product.

Formulations suitable for parenteral administration include aqueous and non-aqueous sterile injection solutions which may contain anti-oxidants, buffers, bacteriostats and solutes which render the formulation appropriate for the intended recipient; and aqueous and non-aqueous sterile suspensions which may include suspending agents and thickening agents. The formulations may be presented in unit-dose or multi-dose containers, for example sealed ampules, vials or syringes, and may be stored in a freeze-dried (lyophilised) condition requiring only the addition of the sterile liquid carrier, for example water for injections, immediately prior to use. Extemporaneous injection solutions and suspensions may be prepared from sterile powders. Dosage formulations may contain the Therapeutic protein portion at a lower molar concentration or lower dosage compared to the non-fused standard formulation for the Therapeutic protein given the extended serum half-life exhibited by many of the albumin fusion proteins of the invention.

As an example, when an albumin fusion protein of the invention comprises one or more of the Therapeutic protein regions, the dosage form can be calculated on the basis of the potency of the albumin fusion protein relative to the potency of the Therapeutic protein, while taking into account the prolonged serum half-life and shelf-life of the albumin fusion proteins compared to that of the native Therapeutic protein. For example, in an albumin fusion protein consisting of a full length HA fused to a full length Therapeutic protein, an equivalent dose in terms of units would represent a greater weight of agent but the dosage frequency can be reduced.

Formulations or compositions of the invention may be packaged together with, or included in a kit with, instructions or a package insert referring to the extended shelf-life of the albumin fusion protein component. For instance, such instructions or package inserts may address recommended storage conditions, such as time, temperature and light, talking into account the extended or prolonged shelf-life of the albumin fusion proteins of the invention. Such instructions or package inserts may also address the particular advantages of the albumin fusion proteins of the inventions, such as the ease of storage for formulations that may require use in the field, outside of controlled hospital, clinic or office conditions. As described above, formulations of the invention may be in aqueous form and may be stored under less than ideal circumstances without significant loss of therapeutic activity.

The invention also provides methods of treatment and/or prevention of diseases or disorders (such as, for example, any one or more of the diseases or disorders disclosed herein) by administration to a subject of an effective amount of an albumin fusion protein of the invention or a polynucleotide encoding an albumin fusion protein of the invention (“albumin fusion polynucleotide”) in a pharmaceutically acceptable carrier.

Effective dosages of the albumin fusion protein and/or polynucleotide of the invention to be administered may be determined through procedures well known to those in the art which address such parameters as biological half-life, bioavailability, and toxicity, including using data from routine in vitro and in vivo studies such as those described in the references in Table 1, using methods well known to those skilled in the art.

The albumin fusion protein and/or polynucleotide will be formulated and dosed in a fashion consistent with good medical practice, taking into account the clinical condition of the individual patient (especially the side effects of treatment with the albumin fusion protein and/or polynucleotide alone), the site of delivery, the method of administration, the scheduling of administration, and other factors known to practitioners. The “effective amount” for purposes herein is thus determined by such considerations.

For example, determining an effective amount of substance to be delivered can depend upon a number of factors including, for example, the chemical structure and biological activity of the substance, the age and weight of the patient, the precise condition requiring treatment and its severity, and the route of administration. The frequency of treatments depends upon a number of factors, such as the amount of albumin fusion protein or polynucleotide constructs administered per dose, as well as the health and history of the subject. The precise amount, number of doses, and timing of doses will be determined by the attending physician or veterinarian.

Albumin fusion proteins and polynucleotides of the present invention can be administered to any animal, preferably to mammals and birds. Preferred mammals include humans, dogs, cats, mice, rats, rabbits sheep, cattle, horses and pigs, with humans being particularly preferred.

As a general proposition, the albumin fusion protein of the invention will be dosed lower (on the molar basis of the unfused Therapeutic protein) or administered less frequently than the unfused Therapeutic protein. A therapeutically effective dose may refer to that amount of the compound sufficient to result in lower HIV titers in vivo, amelioration of symptoms or disease stabilization or a prolongation of survival in a patient or improvement in quality of life.

The albumin fusion proteins of the invention are advantageous in that they can simulate continuous infusion of “classic drugs”, i.e., less protein equivalent is needed for identical inhibitory activity.

The albumin fusion proteins of the invention have the following additional advantages: (i) dose optimization design on the basis of the phenotype of the HIV infection to fit specific growth, virus load or resistance characteristics of the HIV (e.g. fast and slow growing); and (ii) controlling/maintaining drug concentration in the efficacous concentration during the duration of therapy. Furthermore, when peptides (such as T-20 and T-1249) are hydrophobic in nature, their fusion to albumin improves their solubility which should also result in an increase of bioavailability and should allow for higher concentrated formulations.

For example, in clinical trials for unfused T-20, a typical dose of 50 mg twice daily was employed (Zhang 2002, Kilby 2002, Kilby 1998) and, in clinical trials of unfused T-1249, dose of 12.5 mg/day to 200 mg/day was employed, which conferred dose-related suppression of HIV (Gulick, 2002). It is expected that the dosage and/or dosing frequency of the T-20 or T-1249 (i.e., the molar equivalent of the active moiety) in the albumin fusion protein of the invention will be less than that of the unfused T-20 or T-1249.

Albumin fusion proteins and/or polynucleotides can be are administered orally, rectally, parenterally, intracisternally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any. The term “parenteral” as used herein refers to modes of administration which include intravenous, intramuscular, intraperitoneal, intrasternal, subcutaneous and intraarticular injection and infusion.

Albumin fusion proteins and/or polynucleotides of the invention are also suitably administered by sustained-release systems such as those described in U.S. Provisional Application Ser. No. 60/355,547 and WO 01/79480 (pp. 129-130), which are incorporated herein by reference.

For parenteral administration, in one embodiment, the albumin fusion protein and/or polynucleotide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation optionally does not include oxidizing agents and other compounds that are known to be deleterious to the Therapeutic.

The albumin fusion proteins and/or polynucleotides of the invention may be administered alone or in combination with other therapeutic agents. Albumin fusion protein and/or polynucleotide agents that may be administered in combination with the albumin fusion proteins and/or polynucleotides of the invention include, but are not limited to, antiretroviral agents like protease, reverse transcriptase, integrase and assembly inhibitors, chemotherapeutic agents, antibiotics, steroidal and non-steroidal anti-inflammatories, conventional immunotherapeutic agents, and/or therapeutic treatments as described, e.g., in U.S. Provisional Application Ser. No. 60/355,547 and WO 01/79480 (pp. 132-151) which are incorporated by reference herein. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

Pharmaceutical compositions suitable for use in the present invention include compositions wherein the active ingredients are contained in an effective amount to achieve its intended purpose.

In certain embodiments, albumin fusion proteins and/or polynucleotides of the invention are administered in combination with antiretroviral agents, nucleoside/nucleotide reverse transcriptase inhibitors (NRTIs), non-nucleoside reverse transcriptase inhibitors (NNRTIs), and/or protease inhibitors (PIs).

The invention also provides a pharmaceutical pack or kit comprising one or more containers filled with one or more of the ingredients of the pharmaceutical compositions comprising albumin fusion proteins of the invention. Optionally associated with such container(s) can be a notice in the form prescribed by a governmental agency regulating the manufacture, use or sale of pharmaceuticals or biological products) which notice reflects approval by the agency of manufacture, use or sate for human administration.

Having generally described the invention, the same will be more readily understood by reference to the following examples, which are provided by way of illustration and are not intended as limiting.

Without further description, it is believed that one of ordinary skill in the art can, using the preceding description and the following illustrative examples, make and utilize the alterations detected in the present invention and practice the claimed methods. The following working examples therefore, specifically point out certain embodiments of the present invention, and are not to be construed as limiting in any way the remainder of the disclosure.

### EXAMPLES

### Example 1

### Construction of N-Terminal and C-Terminal Albumin-(GGS)4GG Linker Cloning Vectors

The recombinant albumin expression vectors pDB2243 and pDB2244 have been described previously in patent application WO 00/44772. The recombinant albumin expression vectors pAYE645 and pAYE646 have been described previously in UK patent application 0217033.0. Plasmid pDB2243 was modified to introduce a DNA sequence encoding the 14 amino acid polypeptide linker N-GGSGGSGGSGGSGG-C ((GGS)₄GG, “N” and “C” denote the orientation of the polypeptide sequence) at the C-terminal end of the albumin polypeptide in such a way to subsequently enable another polypeptide chain to be inserted C-terminal to the (GGS)₄GG linker to produce a C-terminal albumin fusion in the general configuration, albumin-(GGS)₄GG-polypeptide. Similarly, plasmid pAYE645 was modified to introduce a DNA sequence encoding the (GGS)₄GG polypeptide linker at the N-terminal end of the albumin polypeptide in such a way to subsequently enable another polypeptide chain to be inserted N-terminal to the (GGS)₄GG linker to produce an N-terminal albumin fusion in the general configuration of polypeptide-(GGS)₄GCG-albumin.

Plasmid pDB2243, described by Sleep, D., et al. (1991) Bio/Technology 9, 183-187 and in patent application WO 00/44772 which contained the yeast PRB1 promoter and the yeast ADH1 terminator providing appropriate transcription promoter and transcription terminator sequences. Plasmid pDB2243 was digested to completion with BamHI, the recessed ends were blunt ended with T4 DNA polymerase and dNTPs, and finally religated to generate plasmid pDB2566.

A double stranded synthetic oligonucleotide linker Bsu36I/HindIII linker was synthesised by annealing the synthetic oligonucleotides JH033A and JH033B.

JH033A

(SEQ ID NO:12)

5′-TTAGGCTTAGGTGGTTCTGGTGGTTCCGGTGGTTCTGGTGGATCCGG
TGGTTAATA-3′
JH033B

(SEQ ID NO:13)

5′-AGCTTATTAACCACCGGATCCACCAGAACCACCGGAACCACCAGAAC
CACCTAAGCC-3′

The annealed Bsu36I/HindIII linker was ligated into HindIII/Bsu36I cut pDB2566 to generate plasmid pDB2575X wlich comprised an albumin coding region with a (GGS)₄GG peptide linker at its C-terminal end.

Plasmid pAYE645 that contained the yeast PRB1 promoter and the yeast ADH1 terminator providing appropriate transcription promoter and transcription terminator sequences is described in UK patent application 0217033.0. Plasmid pAYE645 was digested to completion with the restriction enzyme AflII and partially digested with the restriction enzyme HindIII and the DNA fragment comprising the 3′ end of the yeast PRB1 promoter and the rHA coding sequence was isolated. Plasmid pDB2241 described in patent application WO 00/44772, was digested with AflII/HindIII and the DNA fragment comprising the 5′ end of the yeast PRB1 promoter and the yeast ADH1 terminator was isolated. The AflII/HindIII DNA fragment from pAYE645 was then cloned into the AflII/HindIII pDB2241 vector DNA fragment to create the plasmid pDB2302. Plasmid pDB2302 was digested to completion with PacI/XhoI and the 6.19 kb fragment isolated, the recessed ends were blunt ended with T4 DNA polymerase and dNTPs, and religated to generate plasmid pDB2465. Plasmid pDB2465 was linearised with ClaI, the recessed ends were blunt ended with T4 DNA polymerase and dNTPs, and religated to generate plasmid pDB2533. Plasmid pDB2533 was linearised with BlnI, the recessed ends were blunt ended with T4 DNA polymerase and dNTPs, and religated to generate plasmid pDB2534. Plasmid pDB2534 was digested to completion with BmgBI/BglII, the 6.96 kb DNA fragment isolated and ligated to one of two double stranded oligonucleotide linkers, VC053/VC054 and VC057/VC058 to create plasmid pDB2540, or VC055/VC056 and VC057/VC058 to create plasmid pDB2541.

VC053

(SEQ ID NO:14)

5′-GATCTTTGGATAAGAGAGACGCTCACAAGTCCGAAGTCGCT
CACCGGT-3′
VC054

(SEQ ID NO:15)

5′-pCCTTGAACCGGTGAGCGACTTCGGACTTGTGAGCGTCTCTCTTATC
CAAA-3′
VC055

(SEQ ID NO:16)

5′-GATCTTTGGATAAGAGAGACGCTCACAAGTCCGAAGTCGCTCATCG
AT-3′
VC056

(SEQ ID NO:19)

5′-pCCTTGAATCGATGAGCGACTTCGGACTTGTGAGCGTCTCTCTTATC
CAAA-3′
VC057

(SEQ ID NO:20)

5′-pTCAAGGACCTAGGTGAGGAAAACTTCAAGGCTTTGGTCTTGATCGC
TTTCGCTCAATACTTGCAACAATGTCCATTCGAAGATCAC-3′
VC058

(SEQ ID NO:21)

5′-GTGATCTTCGAATGGACATTGTTGCAAGTATTGAGCGAAAGCGATCA
AGACCAAAGCCTTGAAGTTTTCCTCACCTAGGT-3′

A double stranded synthetic oligonucleotide linker BglII/AgeI linker was synthesised by annealing the synthetic oligonucleotides JH035A and JH035B.

JH035A

(SEQ ID NO:22)

5′-GATCTTTGGATAAGAGAGGTGGATCCGGTGGTTCCGGTGGTTCTGGT
GGTTCCGGTGGTGACGCTCACAAGTCCGAAGTCGCTCA-3′
JH035B

(SEQ ID NO:23)

5′-CCGGTGAGCGACTTCGGACTTGTGAGCGTCACCACCGGAACCACCAG
AACCACCGGAACCACCGGATCCACCTCTCTTATCCAAA-3′

The annealed BglII/AgeI linker was ligated into BglII/AgeI cut pDB2540 to generate plasmid pDB2573X, which comprised an albumin coding region with a (GGS)₄GG peptide linker at its N-terminal end.

### Example 2

### Construction of N-Terminal and C-Terminal Albumin-T-1249 Fusions

Construction of N-Terminal T-1249-(GGS)₄GG-Albumin Expression Plasmid

A DNA clone comprising the amino acid sequence of T-1249 was generated by joining two synthetic DNA fragments each made from two overlapping synthetic oligonucleotides. DNA fragment 1 was generated by annealing oligonucleotides 5′-GTGAGATCTTTGGATAAGAGATGGCAAGAATGGGAACAAAAGATTAC-3′ (SEQ ID NO:24) and 5′-CACGAGCTTGTTCCAACAAAGCAGTAATCTTTTGTTCCCATTC-3′(SEQ ID NO: 25) and then performing a primer extension reaction with Taq DNA polymerase to create a double-stranded DNA fragment. A similar procedure was performed to create DNA fragment 2, using oligonucleotides 5′-GTGAGCTCAAATTCAACAAGAAAAGAACGAATACGAATTGCAAAAGTTGGA CAAGTGGG-3′ (SEQ ID NO:26) and 5′-CACGGATCCACCGAACCATTCCCACAAAGAAGCCCACTTGTCCAACTTTTGC AATTCGTATTC-3′ (SEQ ID NO:27). Subsequently DNA fragment 1 was digested with restriction endonucleases BglII/AluI and DNA fragment 2 was digested with restriction endonucleases AluI/BamHI. Both fragments were than ligated into vector pLITMUS29 (New England Biolabs), digested with BglII and BamHI to create pLIT-T-1249-N.

Plasmid pLIT-T-1249-N was digested to completion with BamHI and BglII. The 0.14 kb DNA fragment was ligated into BamHI, BglII digested pDB2573 to create plasmid pDB2667. Appropriate yeast vector sequences were provide by a “disintegration” plasmid pSAC35 generally disclosed in EP-A-286 424 and described by Sleep, D., et al. (1991) Bio/Technology 9, 183-197. Plasmid pDB2667 was digested to completion with NotI and the 3.15 kb N terminal T-1249-(GGS)₄GG-rHA expression cassette isolated and subsequently ligated into NotI calf intestinal phosphatase treated pSAC35 to create plasmid pDB2681.

### Construction of C-Terminal Albumin-(GGS)₄GG-T-1249 Expression Plasmid

A PCR fragment was amplified from pLIT-T-1249-N using forward primer 5′-GTGGGATCCGGTGGTTGGCAAGAATGGGAACAAAAGATTAC-3′ (SEQ ID NO:28) and reverse primer 5′-CACAAGCTTATTAGAACCATTCCCACAAAGAAGC-3′ (SEQ ID NO:29). The fragment was digested to completion with BamHI and HindIII and ligated into vector pLITMUS29 similarly digested with BamHI and HindIII to create pLIT-T-1249-C. Plasmid pDB2575 was partially digested with HindIII and then digested to completion with BamHI. The desired 6.55 kb DNA fragment was isolated and ligated with the 0.13 kb BamHI/HindIII fragment from plasmid pLIT-T-1249-C to create plasmid pDB2668.

Appropriate yeast vector sequences were provide by a “disintegration” plasmid pSAC35 generally disclosed in EP-A-286 424 and described by Sleep, D., et al. (1991) Bio/Technology 9, 183-187. Plasmid pDB2668 was digested to completion with NotI and the 3.151 kb C terminal rHA-(GGS)₄GG-T-1249 expression cassette isolated and subsequently ligated into NotI calf intestinal phosphatase treated pSAC35 to create plasmid pDB2682.

### Example 3

### Construction of N-Terminal and C-Terminal Albumin-T-20 Fusions

Generation of the Basic Clone

Cloning of the sequence of T-20 was performed by amplification of a PCR fragment by RT-PCR on RNA isolated from a HIV-1 containing cell culture supernatant, using forward primer 5′-GTGCCTTGGAATGCTAGTTG-3′ (SEQ ID NO:30) and reverse primer 5′-CTTAAACCTACCAAGCCTCC-3′ (SEQ ID NO:31) and subsequent cloning into vector pCR4-TOPO (Invitrogen) to create pCR4-HIV-T-20.

Construction of N-Terminal T-20-(GGS)₄GG-Albumin Expression Plasmid

A PCR fragment was amplified from pCR4-HIV-T-20 using the forward primer DS223 5′-CTCTAGATCTTTGGATAAGAGATACACCAGCTTAAIACACTCCTTAATTGAA G-3-(SEQ ID NO:32) and reverse primer DS224 5′-CCACCGGATCCACCAAkACCAATTCCACAAACTTGCCCATTTATC-3′ (SEQ ID NO:33). The DNA fragment was digested to completion with BglII and BamHI and the 0.13 kb DNA fragment and ligated into pDB2573 similarly digested with BglII and BamHI to create pDB2593. Appropriate yeast vector sequences were provide by a “disintegration” plasmid pSAC35 generally disclosed in EP-A-286 424 and described by Sleep, D., et al. (1991) Bio/Technology 9, 193-187. The NotI N-terminal T-20-(GGS)₄GG-rHA expression cassette was isolated from pDB2593, purified and ligated into NotI digested pSAC35 which had been treated with calf intestinal phosphatase, creating two plasmids; the first pDB2595 contained the NotI expression cassette in the same expression orientation as LEU2, while the second pDB2596 contained the NotI expression cassette in the opposite orientation to LEU2.

Construction of C-Terminal Albumin-(GGS)₄GG-T-20-Expression Plasmid

A PCR fragment was amplified from pCR4-HIV-T-20 using the forward primer DS225 5′-TGGTGGATCCGGTGGTTACACCAGCTTAATACACTCCTTAATTGAAGAATCG C-3′ (SEQ ID NO:34) and reverse primer DS226 5′ AATTAAGCTTATTAAAACCAATTCCACAAACTTGCCCATTTATCTAATTCC-3′ (SEQ ID NO:35). The DNA fragment was digested to completion with BamHI and HindIII and the 0.13 kb DNA fragment and ligated into pDB2575 similarly digested with BamHI and HindIII to create pDB2594. Appropriate yeast vector sequences were provide by a “disintegration” plasmid pSAC35 generally disclosed in EP-A-286 424 and described by Sleep, D., et al. (1991) Bio/Technology 9, 183-187. The NotI C-terminal rHA-(GGS)₄GG-T-20 expression cassette was isolated from pDB2594, purified and ligated into NotI digested pSAC35 which had been treated with calf intestinal phosphatase, creating two plasmids; the first pDB2597 contained the NotI expression cassette in the same expression orientation as LEU2, while the second pDB2598 contained the NotI expression cassette in the opposite orientation to LEU2.

### Example 4

### Yeast Transformation and Culturing Conditions

Yeast strains disclosed in WO 95/23857, WO 95/33833 and WO 94/04687 were transformed to leucine prototrophy as described in Sleep D., et al. (2001) Yeast 18, 403-421. The transformants were patched out onto Buffered Minimal Medium (Ban, described by Kerry-Williams, S. M. et al. (1998) Yeast 14, 161-169) and incubated at 30° C. until grown sufficiently for further analysis.

### Example 5

### Expression and Purification of Albumin T-20 Fusion Proteins

rHA fusions were expressed in shake flask culture and the expression levels were measured by SDS-PAGE using an albumin standard. The expression level in fermentation culture (as described in WO 00/44772) supernatant was >2 g·L⁻¹ for both rHA-GS-T-20 and T-20-GS-rHA.

### C-Terminal T-20 Purification

The C-Terminal T-20 was purified using the standard rHA SP-FF conditions and elution buffer as described in WO 00/44772. The eluate was then purified using standard rHA DE-FF conditions, except that an extra 200 mM NaCl was used in the elution buffer (although this salt concentration was not optimized and, therefore, may be varied). The purified material was then concentrated and diafiltered against PBS.

### N-Terminal T-20 Purification

The N-Terminal T-20 was purified using the standard rHA SP-FF conditions and elution buffer. The eluate was then purified using standard rHA DE-FF conditions. The DE-FF was eluted using both the standard elution buffer and also standard elution containing 200 mM NaCl. The two eluates appeared different by SDS-PAGE and were processed separately as DE-FF Eluate #1 and DE-FF Eluate #2. The two purified materials were then concentrated to >5 mg/mL and diafiltered against 7 continuous volumes of PBS using 10 kDa molecular weight cut-off membranes.

### Example 6

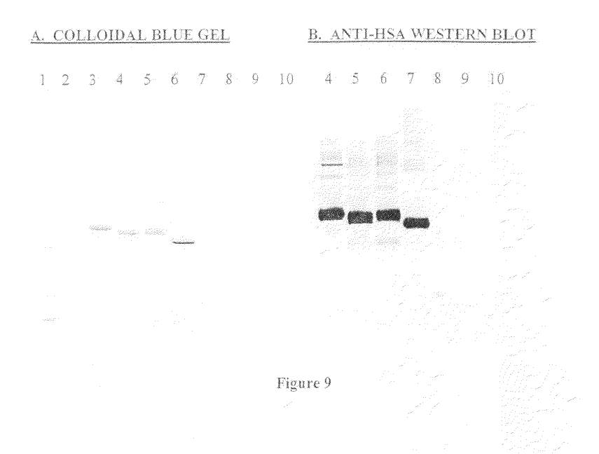

The purified T-20 albumin fusion proteins were characterized by removing the samples on a 4-12% gradient SDS non-reducing gel and performing a Western blot with anti-HSA antibodies. The results are shown in FIG. 9. Legend: (A) Colloid as Blue Gel: (B) anti-HSA Western blot. The samples were loaded as follows:

| Lane | Sample | Load | |
|---|---|---|---|
| 1. | Magic Marker | — | |
| 2. | — | — | |
| 3. | C Terminal T-20 | 1 | μg |
| 4. | N Terminal T-20 Eluate #1 | 1 | μg |
| 5. | N Terminal T-20 Eluate #2 | 1 | μg |
| 6. | HSA | 1 | μg |
| 7. | — | — | |
| 8. | — | — | |
| 9. | SPT9901* | 100 | ng |

| | | |
|---|---|---|
| 10. | — | — |

*“SPT9901” is a yeast fermentation culture supernatant which does not contain albumin or an albumin-fusion. It is used to show that an immunological cross-reactivity detected on the western blot is due to some specific species produced by the albumin-fusion expression yeast strain, rather than a non-specific cross-reactivity between the antibody and a yeast (host) derived component.

### Example 7

### Pharmacokinetics of Albumin T-20 Fusion Proteins

### Animal Model

Three male and three female rabbits per group received albumin-fused T-20 (350 μg/kg) by a single i.v. or s.c. injection on day 0. Blood samples were drawn for the determination of the antigen levels at baseline and at 5 min, 10 min, 20 min, 30 nm in, 45 min, 1 h, 2 h, 4 h, 8 h, 24 h (1 d), 48 h (2 d), 72 h (3 d), 5 d, 7 d, 9 d, 11 d, and 14 d after i.v. administration of the test substance and at baseline, 30 min, 1 h, 2 h, 4 h, 8 h, 24 h (1 d), 48 h (2 d), 72 h (3 d), 5 d, 7 d, 9 d, 11 d and 14 d following s.c. injection.

### Variables

### Pharmacokinetic (PK) Variables:

Area under the plasma concentration time curve (AUC), maximum concentration (Cₘₐₓ), time of maximum concentration (tₘₐₓ) mean residence time, half-lives of absorption and distribution (if applicable), clearance, volume of distribution.

### Analytical Methods

Albumin-fused T-20 plasma concentration was determined with an anti-human albumin ELISA. Albumin-fused T-20 served for generation of the standard curve. The detection limit of the ELISA was 5 ng/mL.

### Statistical Methods

### Analysis of Individual Plasma Levels

The plasma concentration-time profiles of albumin-fused T-20 were analyzed per animal by means of nonlinear regression. Values reported as <5 ng/n-L were treated as 5 ng/mL. The value measured at baseline was subtracted from the subsequent concentrations. Any resulting negative values were set to zero. An exponential model was fitted to the data by the method of least squares. For the profiles following i.v. administration, an open two-compartment model was used. For the profiles following s.c. administration, an open one-compartment model with first-order input and lag-time was used.

In the s.c. treated group, the pharmacokinetic analysis was confined to the plasma levels of the first seven days because afterwards presumably antibody formation rendered the determination of plasma levels unreliable.

In the i.v. treated group, the AUC was calculated using the linear trapezoidal rule up to the last measured value (AUC_{0-14d}).

Due to the restriction to the first seven days in the s.c. group, the AUC up to Day 7 (AUC_{0-7d}) was calculated in addition for both groups and instead of AUC_{0-7d} in the s.c. group.

### Summary and Comparative Analyses

Individual PK results were summarized descriptively per route of application (minimum, median, maximum, mean, standard deviation).

A two-way analysis of variance was carried out for the primary variables: elimination half-life, AUC and Cₘₐₓ(all log-transformed). Fixed factor was the route of administration. Appropriate contrasts between treatment groups were evaluated. The possibility of unequal variances was also taken into account.

For the purpose of this analysis it was assumed that ln (half-life), ln (AUC) and ln (Cₘₐₓ) each follow a normal distribution.

Bioavailabilities were compared between routes of administration (absolute bioavailability) at an alpha level of 0.1 using a two-sided 90% confidence interval.

### Results

The means and standard deviations of the T-20-AFP concentrations at every time point are shown in FIG. 13.

In the animals treated intravenously, the T-20-AFP levels showed a distribution phase of about 10 hours, followed by a slower elimination phase. The levels stayed above 100 ng/mL throughout the 14-day period.

In the animals treated subcutaneously, the levels appeared in plasma after a mean lag-time of 2.3 hours. They reached their peak between 24 and 48 hours after injection. For 7 days, the decline curve was practically identical to that of the i.v. group. After that point, the levels dropped sharply, possibly indicating the interference of antibodies. The values measured after more than 7 days were therefore not included in the pharmacokinetic analysis.

### Conclusion

T-20-AFP was eliminated from plasma with an average terminal half-life of 81 hours after i.v. application and 76 hours after s.c. application. The half-lives in the two groups were in very good agreement, the difference was not statistically significant.

After i.v. application, a distribution phase lasting about 10 hours with a mean half-life of 4.1 hours preceded the elimination phase. After s.c. administration, following an average lag-time of 2.3 hours, T-20-AFP levels rose with a mean absorption half-life of 7.9 hours.

The bioavailability after s.c. application with respect to the AUC from 0 to 7 days was 72%, with respect to Cₘₐₓ it was 28%

These data demonstrate the capability of the albumin to extend plasma half-life of the T-20 peptide significantly. Terminal half-life of the T-20 peptide in humans was reported to be 3.46 and 4.35 hours for the 45 and 180 mg subcutaneous dose, respectively (Zhang X et al., 2002). Together with the data described above obtained in rabbits, it is estimated that a plasma half-life extension of the T-20 peptide fused to albumin by at least 10-fold is achievable in humans.

### Example 8

### In Vitro Efficacy of Albumin T-20 Fusion Proteins

Antiviral activities of HIV peptide albumin fusions were tested in vitro in a cell-cell fusion assay. Basically, a Jurkat cell suspension (2×10⁶/ml) was mixed with HIV inhibitor or control protein solution and HIV-1 (1.5×10⁵/ml), each sample being assayed in 8 replicates, and incubated at 37° C. for 4 days. Each sample was evaluated for amount and quality of cell-cell fusions, antiviral activity being measured through reduction of fusion events compared to control. Both purified protein and yeast cell culture supernatant were suitable for the assay.

### T-20 Produced in PMT1 Strain

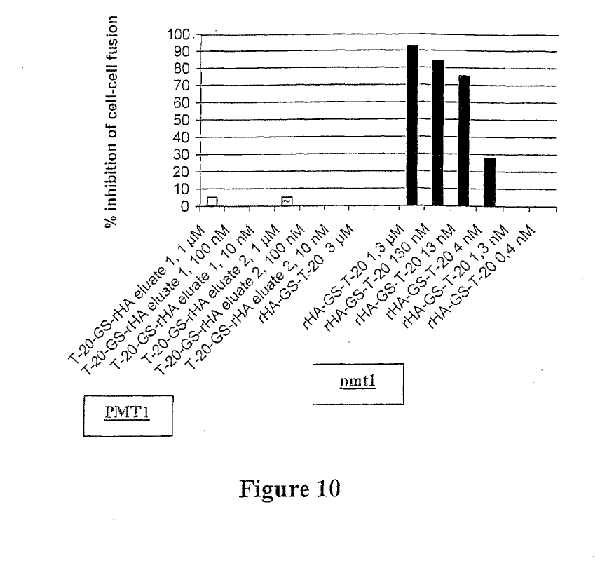

rHA-GS-T-20 and T-20-GS-rHA, produced in the standard yeast strain, were purified and screened in the above test for antiviral activity. Controls included medium control (RPMI), 1 and 3 μM recombinant albumin. RHA-GS-T-20 was used in a 3 μM concentration in which it did not display any HIV inhibitory effect and the average number of fusion events was comparable to the Recombumin control. These results are shown in FIG. 10. Similar results were obtained with purified T-20-GS-rHA (2 eluates, see Example 6), but here at least the 1 μM concentration showed a slight antiviral effect. The differences in O-linked mannosylation might be a likely explanation for the absent or low anti-HIV activity of rHA-GS-T-20 and T-20-GS-rHA, respectively. This assumption is supported by the following experiments.

T-20 Produced in pmt1 Strain

To reduce O-linked glycosylation, a phosphomannosyl transferase 1 gene deficient (pmt1) yeast strain was used for expression of rHA-GS-T-20 and now significant antiviral activity could be observed. The 50% inhibitory concentration was between 4 and 13 nM (FIG. 10), suggesting antiviral activity in a similar range to the T-20 peptide (Wild C T et al. 1994. PNAS 91:9770-9774).

### Example 9

### Expression and Purification C-Terminal T-1249 Albumin Fusion Protein

rHA fusion was expressed in shake flask culture and the expression levels were measured by SDS-PAGE using an albumin standard. The expression level in fermentation culture (performed as described in WO 00/44772, at pH 5.5) supernatant was >2 g·L⁻¹ for rHA-GS-T-1249.

The C-Terminal T-1249 was purified using the standard rHA SP-FF conditions and elution buffer described in WO 00/44772. The eluate was then purified using standard rHA DE-FF conditions described in WO 00/44772, except the standard rHA elution was used as a wash with the fraction discarded and an additional elution performed containing an extra 200 mM NaCl in the standard rHA elution buffer. The purified material was then concentrated to >5 mg/mL and diafiltered against 7 continuous volumes of PBS using 10 kDa molecular weight cut-off membranes.

### Example 10

### Characterization of C-Terminal T-1249 Albumin Fusion Protein After Purifications

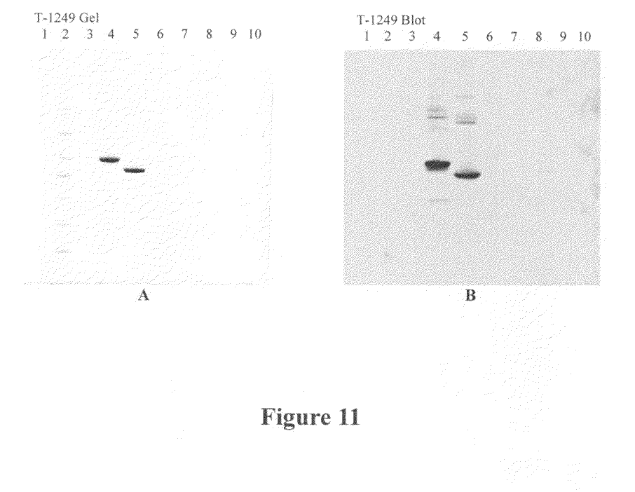

The purified C-Terminal T-1249 albumin fusion protein was characterized by removing the samples on a 4-12% gradient SDS non-reducing gel and performing a Western blot with anti-HSA antibodies. The results are shown in FIG. 11. Legend: (A) Colloid as Blue Gel; (B) anti-HSA Western blot. The samples were loaded as follows:

| Lane | Sample | Load | |
|---|---|---|---|
| 1. | — | — | |
| 2. | Magic marker 1/10 | 5 | μL |
| 3. | — | — | |
| 4. | T-1249 C-Terminal | 1 | μg |
| 5. | HSA | 1 | μg |
| 6. | — | — | |
| 7. | — | — | |
| 8. | SPT9901 | 100 | ng |

| | | |
|---|---|---|
| 9 | — | — |
| 10 | — | — |

### Example 11

### In Vitro Efficacy of Albumin T-1249 Fusions After Purification

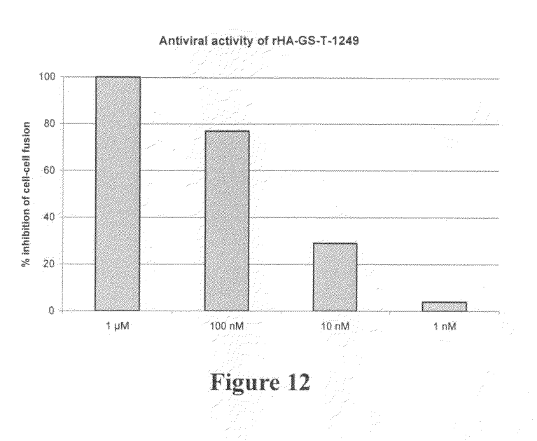

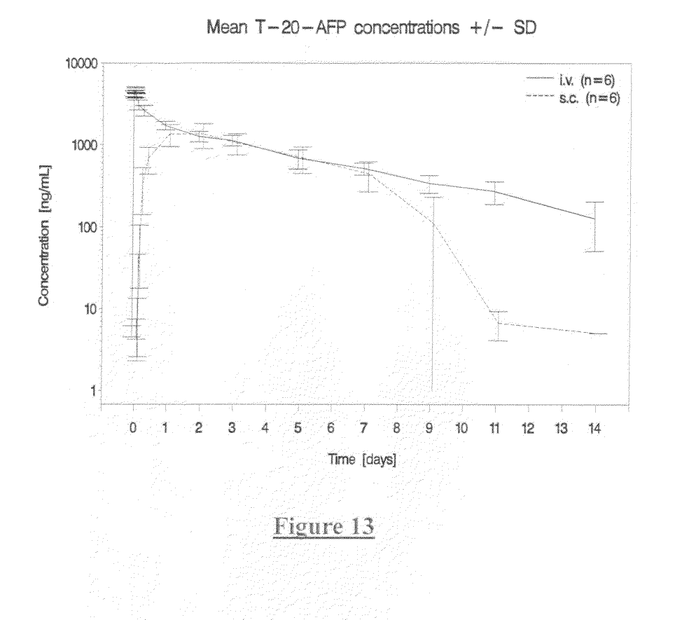

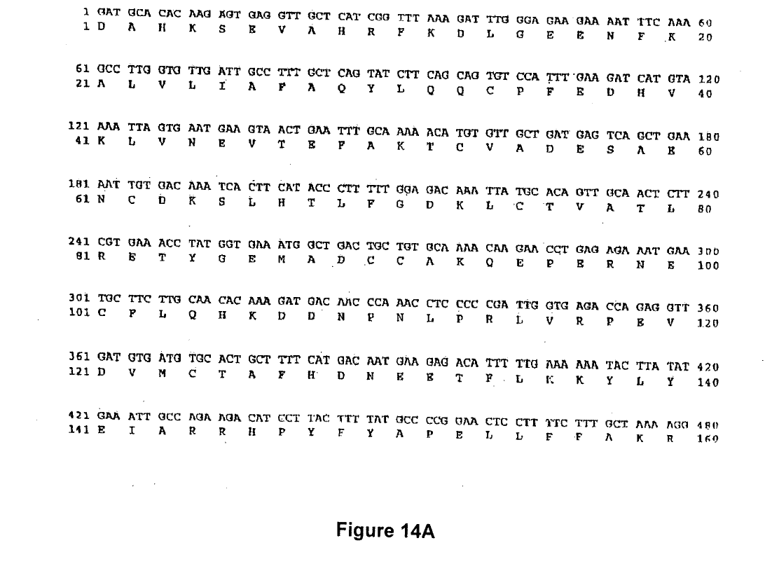

T-1249-GS-rHA, produced in the standard (PMT1) yeast strain, was purified (Example 9) and tested in the above assay for antiviral activity. FIG. 12 displays the results which show that a 50% inhibitory concentration (TC₅₀) was obtained between 10 and 100 nM.

### REFERENCES

Boyd, M. R. et al., “Discovery of Cyanovirin-N, a Novel Human Immunodeficiency Virus-Inactivating Protein That Binds Viral Surface Envelope Glycoprotein gp120: Potential Applications to Microbicide Development”, Antimicrobial Agents and Chemotherapy, 41:1521-15, 0 (1997)
Chan, D. C. et al., “Evidence that a prominent cavity in the coiled coil of HIV type 1 gp41 is an attractive drug target”, Proc. Natl. Acad. Sci. USA, 95:15613-15617 (1998)
Chan, D. C. et al., “HIV entry and its inhibition”, Cell, 93:681-684; (1998)
Church, J. A. et al., “Safety and antiretroviral activity of chronic subcutaneous administration of T-20 in human immunodeficiency virus 1-infected children”, The Pediatric Infectious Disease Journal, 21:653-659 (2002)
Cohen, C. J. et al., “Long-Term Treatment with Subcutaneous T-20, a Fusion Inhibitor, in HIV-infected Patients: Patient Satisfaction and Impact on Activities of Daily Living”, AIDS Patient Care and STDs, 16:327-335 (2002)
D'Souza, M. P. et al., “Current Evidence and Future Directions for Targeting HIV Entry”, JAMA, 284:215-222 (2000)
Doranz, B. J., “The use of Envelope for HIV therapeutics: from vaccines to co-receptors”, Emerging Therapeutic Targets, 4:423-437 (2000)
Eckert, D. M. et al., “Design of potent inhibitors of HIV-1 entry from the gp41 N-peptide region”, PNAS, 98:11187-11192 (2001)
Eckert, D. M. et al., “Inhibiting HIV-1 Entry: Discovery of D-Peptide Inhibitors that Target the gp41 Coiled-Coil Pocket”, Cell, 99:103-115 (1999)
Esté, J. A., “HIV Resistance to Entry Inhhibitors”, AIDS Reviews, 3:121-132 (2001)
Greenberg, M. L., “Enfuvirtide (T-20) and T-1249 resistance: observations from phase II clinical trials of enfuvirtide in combination with oral antiretrovirals and a phase I/II dose-ranging monotherapy trial of T-1249”, Antiviral Ther., 7:S106-S107 (2002)
Gulick, R. et al., “Complete analysis of T-1249-101: Safety, pharmacokinetics and antiviral activity of T-1249, a peptide inhibitor of 111V membrane fusion”, Program and abstracts of the 42^{nd} Interscience Conference on Antimicrobial Agents and Chemotherapy, Sep. 27-30, 2002, San Diego, Calif. Abstract H-1075.
Gustafson, K. R. et al., “Isolation, Primary Sequence Determination, and Disulfide Bond Structure of Cyanovirin-N, an Anti-HIV (Human Immunodeficiency Virus) Protein from the Cyanobacterium Nostoc ellipsosporum”, Biochemical and Biophysical Research Communications, 238:223-228 (1997)
Hanna, S. L. et al., “Resistance mutation in HIV entry inhibitors”, AIDS, 16:1603-1608 ((2002)
Kilby, J. M. et al., “Potent suppression of HIV-1 replication in humans by T-20, a peptide inhibitor of gp41-mediated virus entry”, Nature Medicine 4:1302-1307 (1998)
Kilby, J. M. et al., “Potent suppression of HIV-1 replication in humans by T-20, a peptide inhibitor of gp41-mediated virus entry”, Nature Medicine, 4:1302-1307 (1998)
Kilby, J. M. et al., “The Safety, Plasma Pharmacokinetics, and Antiviral Activity of Subcutaneous Enfuvirtide (T-20), a Peptide Inhibitor of gp41-Mediated Virus Fusion, in HIV-Infected Adults”, AIDS Research and Human Retroviruses, 18:685-693 (2002)
LaBranche, C. C., “HIV fusion and its inhibition”, Antiviral Research, 50:95-115 (2001)
Lalezari, J. et al., “A controlled phase II trial assessing three doses of T-20 in combination with abacavir, amprenavir, low dose ritonavir and efivarenz in non-nucleoside naïve protease inhibitor experienced HIV-1 infected adults”, Program and abstracts of The 8th Conference on Retroviruses and Opportunistic Infections; Feb. 4-8, 2001; Chicago, Ill. Abstract LB5 (2001)
Qureshi et al., “Characterization of a putative cellular receptor for HIV-1 transmembrane glycoprotein using synthetic peptides”, AIDS, 4:553-558 (1990)
Rimsky L. T. et al., “Potent suppression of HIV-1 replication in humans by T-20, a peptide inhibitor of gp41-mediated virus entry”, Nature Med., 4: 1302-1307 (1998)
Root, M. J. et al., “Protein Design of an HIV-1 Entry Inhibitor”, Science, 291:884-888 (2001)
Wei, X. et al., “Emergence of Resistant Human immunodeficiency Virus Type 1 in Patients Receiving Fusion-Inhibitor (T-20) Monotherapy”, Antimicrobial Agents and Chemotherapy, 46:1896-1905 (2002)
Wild, C. T. et al., “A Synthetic Peptide from HIV-1 gp41 Is a Potent Inhibitor of Virus-Mediated Cell-Cell Fusion”, AIDS Research and Human Retroviruses, 9:1051-1053 (1993)
Wild, C. T. et al., “A synthetic peptide inhibitor of human immunodeficiency virus replication: Correlation between solution structure and viral inhibition”, Proc. Natl. Acad. Sci. USA, 89:10537-10541 (1992)
Wild, C. T. et al., “Peptides corresponding to a predictive a-helical domain of human immunodeficiency virus type 1 gp41 are potent inhibitors of virus infection”, Proc. Natl. Acad. Sci., 91:9770-9774 (1994)
SCRIP Magazine, September 2002, pp. 7-10.
UNAIDS 2001
UNAIDS AIDS Epidemic Update December 2001
Zhang X et al., “Pharmacokinetics of plaza enfuivirtide after subcutaneous administration to patients with human immunodeficiency virus: inverse Gaussian density absorption and 2-compartment disposition”. Clinical Pharmacology and Therapeutics 72:10-19 (2002).
U.S. Pat. No. 5,464,933
U.S. Pat. No. 5,656,480
U.S. Pat. No. 5,821,081
U.S. Pat. No. 5,843,882
U.S. Pat. No. 6,060,065
U.S. Pat. No. 6,068,973
U.S. Pat. No. 6,133,418
U.S. Pat. No. 6,258,782
U.S. Pat. No. 6,348,568
WO 94/28920
WO 99/48513
WO 99/59615
WO 01/03723
WO 01/37896
EP 0652 895

The present invention is not to be limited in scope by the specific embodiments described which are intended as single illustrations of individual aspects of the invention and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

Every reference cited hereinabove is incorporated by reference in its entirety.

What is claimed is:

1. An albumin fusion protein comprising a human immunodeficiency virus (“HIV”) fusion inhibiting peptide, or a fragment or variant thereof, and albumin, or a fragment or variant thereof, wherein the albumin, or fragment or variant thereof, has an albumin activity.

2. The albumin fusion protein of claim 1 comprising HIV env, or a fragment or variant thereof, and albumin, or a fragment or variant thereof.

3. The albumin fusion protein of claim 1 wherein the HIV fusion inhibiting peptide is a peptide which binds to HIV env.

4. The albumin fusion protein of claim 1 wherein the HIV fusion inhibiting peptide is HIV gp41, or a fragment or variant thereof.

5. The albumin fusion protein of claim 1 wherein the HIV fusion inhibiting peptide is a peptide which binds to HIV gp41.

6. The albumin fusion protein of claim 1 wherein the HIV fusion inhibiting peptide is T-20 or T-1249, or a fragment or variant of T-20 or T-1249.

7. The albumin fusion protein of claim 1 wherein the albumin fusion protein comprises at least two HIV fusion inhibiting peptides or fragments or variants thereof.

8. The albumin fusion protein of claim 7 which comprises a first HIV fusion inhibiting peptide, or fragment or variant thereof, and a second HIV fusion inhibiting peptide, or fragment or variant thereof, wherein said first HIV fusion inhibiting peptide, or fragment or variant thereof, is different from said second HIV fusion inhibiting peptide, or fragment or variant thereof.

9. The albumin fusion protein of claim 1 wherein said albumin activity has the ability to prolong the in vivo half-life of the HIV fusion inhibiting peptide, or a fragment or variant thereof, compared to the in vivo half-life of the HIV fusion inhibiting peptide, or a fragment or variant thereof, in an unfused state.

10. The albumin fusion protein of claim 1 further comprising one or more additional HIV fusion inhibiting peptide, or a fragment or variant thereof, or one or more additional albumin, or a fragment or variant thereof.

11. The albumin fusion protein of claim 1 wherein said fusion protein further comprises a chemical moiety.

12. The albumin fusion protein of claim 1 wherein the HIV fusion inhibiting peptide, or fragment or variant thereof, is fused to the N-terminus of albumin, or the N-terminus of the fragment or variant of albumin.

13. The albumin fusion protein of claim 1 wherein HIV fusion inhibiting peptide, or fragment or variant thereof, is fused to the C-terminus of albumin, or the C-terminus of the fragment or variant of albumin.

14. The albumin fusion protein of claim 1 wherein HIV fusion inhibiting peptide, or fragment or variant thereof, is fused to an internal region of albumin, or an internal region of a fragment or variant of albumin.

15. The albumin fusion protein of claim 1 wherein the HIV fusion inhibiting peptide, or fragment or variant thereof, is separated from the albumin or the fragment or variant of albumin by a linker.

16. The albumin fusion protein of claim 1 wherein the albumin fusion protein comprises the following formula:
R2-R1; R1-R2; R2-R1-R2; R2-L-R1-L-R2; R1-L-R2; R2-L-R1; or R1-L-R2-L-R1, wherein R1 is at least one Therapeutic protein, peptide or polypeptide sequence (including fragments or variants thereof), and not necessarily the same Therapeutic protein, L is a linker and R2 is a serum albumin sequence (including fragments or variants thereof).

17. The albumin fusion protein of claim 1 wherein the in vivo half-life of the albumin fusion protein is greater than the in vivo half-life of the HIV fusion inhibiting peptide in an unfused state.

18. The albumin fusion protein of claim 1 wherein the in vitro biological activity of the HIV fusion inhibiting peptide, or fragment or variant thereof, fused to albumin, or fragment or variant thereof, is greater than the in vitro biological activity of the HIV fusion inhibiting peptide, or fragment or variant thereof, in an unfused state.

19. The albumin fusion protein of claim 1 wherein the in vivo biological activity of the HIV fusion inhibiting peptide, or fragment or variant thereof, fused to albumin, or fragment or variant thereof, is greater than the in vivo biological activity of the HIV fusion inhibiting peptide, or fragment or variant thereof, in an unfused state.

20. The albumin fusion protein of claim 1 which is expressed in yeast.
